(19) United States
(12) Patent Application Publication
(10) Pub. No.: US 2026/0345859 A1
(43) Pub. Date: Nov. 5, 2026

(54) SUPRAMOLECULAR HYBRID HYDROGELS FOR BURN AND WOUND DRESSINGS

(71) Applicants: Trustees of Tufts College, Medford, MA (US); The General Hospital Corporation, Boston, MA (US)

(72) Inventors: Ayse Asatekin Alexiou, Arlington, MA (US); Ayse Aslihan Gokaltun, Chestnut Hill, MA (US); Osman Berk Usta, Andover, MA (US); Luca Mazzaferro, Somerville, MA (US); Zosia Stafford, Palo Alto, CA (US)

(21) Appl. No.: 19/103,968

(22) PCT Filed: Sep. 7, 2023
(86) PCT No.: PCT/US2023/032178

§ 371 Date: Feb. 14, 2025

Related U.S. Application Data
(60) Provisional application No. 63/404,327, filed on Sep. 7, 2022.

(51) Int. Cl. A61L 26/00; C08L 33/08; C08L 33/26

(52) U.S. Cl. CPC A61L 26/008; A61L 26/0004; A61L 26/0014; A61L 26/0019; A61L 26/0066; C08L 33/08; C08L 33/26; A61L 2300/104; A61L 2300/406; A61L 2430/34

(57) ABSTRACT

Disclosed are supramolecular hybrid hydrogels (SHH), wound dressings, comprising supramolecular hybrid hydrogels; and methods of treating wounds, including burns.

### RELATED APPLICATIONS

This application claims the benefit of priority to United States Provisional application for Patent Ser. No. 63/404,327, filed Sep. 7, 2022.

### GOVERNMENT SUPPORT

This invention was made with government support under 5R01HL145031, 1R21GM136002, and 1R21GM141683 awarded by the National Institutes of Health. The government has certain rights in the invention.

### BACKGROUND

Burn injuries are devastating and demanding critical care problems and present a major public health predicament, especially in low and middle-income countries^{1,2}. Burn injuries are the fourth most common type of trauma globally and among the most complicated to manage³. Even, in the United States, ~500,000 burn injuries that are severe enough to require treatment occur each year⁴. The direct medical costs for caring for these patients in the US were ~$1.5 billion, and the associated indirect costs with burns exceeded $5 billion in 2013⁵. Various wound dressings exist in the clinic to treat partial-thickness burns⁶. Yet, they are far from ideal, and none of them are considered the “gold standard.” Among these, hydrogel dressings have emerged as a critical alternative to promoting healthy and timely wound healing since they can absorb the wound exudate, provide a moist environment for the wound, and prevent bacterial infections via embedded moieties. However, they adhere to the wound and need to be mechanically or surgically debrided, leading to the traumatization of newly epithelialized tissues, delayed healing, and severe pain for the patient⁷.

Burn dressing removal is reported to be the time of most pain (after the burn), and opioids have, thus, become the cornerstone of burn wound treatment⁸. The average burn dressing change in a typical injury requiring ICU level care takes ~57 minutes with anesthesia and can extend to over 3 hours. For example, it takes three people 138 min to dress for a 10-30% burn, 105 min for a facial burn, and 66 min for a hand dressing change⁹. Anesthesia further increases the time and complexity^{9,10}. Hence, alternative hydrogels that enable easy and pain-free dressing changes while minimizing procedural tissue damage are urgently needed.

On-demand dissoluble hydrogels are a particularly interesting class of wound dressings, as the on-demand dissolution would i) ease the removal of the dressing, ii) require no additional mechanical and/or surgical debridement, iii) reduce dressing change time, and iv) minimize painful procedures. So far, on-demand dissoluble hydrogels have been reported based on chemically crosslinked hydrogels (CCHs)¹¹⁻¹⁸ or physically crosslinked hydrogels (PCHs)¹⁹. CCHs utilize cleavable moieties that can undergo degradation through chemical or enzymatic reactions²⁰. This results in long-dissolution times (≥30 mins)^{11,13-15,21-23} and involves toxic compounds or side-reactions when used in wound care. On the other hand, PCHs can be dissolved via various stimuli (e.g., pH or temperature)²⁴. However, their clinical adoption is hindered by their poor mechanical properties²⁴. Moreover, these traditional approaches comprise complicated processing steps during hydrogel synthesis rendering them unfavorable for large-scale production, and lack in vivo and in vitro data to gauge the biocompatibility and clinical relevance of such hydrogels in burn care^{11,12,19,24}. Although promising progress has been made on on-demand dissoluble hydrogels for burn wound care^{11,12,18,19}, these numerous hurdles have made on-demand dissolution of hydrogels elusive in practice (Table 1). Further research and development are assuredly needed to facilitate the clinical translation and adoption of such hydrogels.

TABLE 1
Comparison of current on-demand dissoluble synthetic hydrogels developed for wound
care and the SHH disclosed herein.

| | | Storage | | | DSA and | | | |
|---|---|---|---|---|---|---|---|---|
| | | Modulus | Self- | Wound | Dissolution | Hydrogel | DSA | Throughput/ |
| Types | Approach | (G′) | Healing | Type | time | Toxicity | Cytotoxicity | Scalability |
| CCHs | Thiol-thioester | 10 kPa | N/A | Burn & | CME | Insufficient | Significant | Very low |
| | exchange¹¹ | | | others | 30 min | in vitro data | toxicity (only | (8-10 steps) |
| | | | | | | & in vivo | tested with | |
| | | | | | | study | fibroblasts) | |
| | Thiol-disulfide | 2 kPa | N/A | Nitrogen | GSH | Insufficient | Not reported, | High |
| | exchange¹⁵ | | | mustard | 30-40 min | in vitro data | likely toxic | (3 steps) |
| | | | | injuries | (1% GSH) | & lack in- | | |
| | | | | | 15-20 min | vivo study | | |
| | | | | | (3% GSH) | | | |
| | | | | | 10-15 min | | | |
| | | | | | (5% GSH) | | | |
| | Selenol | 10 kPa | N/A | N/A | H₂O₂, | Insufficient | Not reported | High |
| | chemistry¹² | | | | 30 min | in-vitro, | | (3 steps) |
| | | | | | | lack in-vivo | | |
| | | | | | | study | | |
| PCHs | Supramolecular | 1.2 kPa | 1 min | N/A | MH | Insufficient | Not reported, | Very low |
| | hydrogel¹⁹ | | | | 2 min (100 | in vitro | likely toxic | (6 steps) |
| | | | | | mM MH) | data, lack | due to high | |
| | | | | | | in-vivo | DSA conc | |
| | | | | | | study | (100 mM) | |
| SHH | Supramolecular | 50 kPa | 1 min | Target: | AH, MH, | No toxicity, | No toxicity | Very high |
| (this | hybrid hydrogel | | | Burn, 2^{nd} | derivatives | >90% | on human | (2 steps) |
| invention) | | | | degree | 4-6 min (w/ | viability in | keratinocytes | |
| | | | | | 20-40 mM | vitro, well | & fibroblasts | |
| | | | | | AH) | tolerated in | | |
| | | | | | | vivo | | |

CME: L-cysteine methyl ester, GSH: Glutathione, MH: Memantine hydrochloride, AH: Adamantine hydrochloride

Supramolecular hydrogels (SHs), a new class of PCHs, are held together by non-covalent crosslinks such as host-guest chemistry, electrostatic interactions, and hydrogen bonding²⁵. These interactions eliminate the need for several synthesis steps and challenging purification procedures²⁶. Host-guest chemistry is a rapidly expanding approach for building SHs where two or more molecules with unique structural connections and noncovalent binding can create complexes. Host-guest chemistry is highly versatile due to its specificity, interaction strength, and controlled reversibility²⁷. Crown ethers, cucurbit[n]urils (CBs), calix[n]arenes, pillar[n]arenes and cyclodextrins (CDs) are macrocyclic molecules used as hosts to encapsulate smaller organic molecules (guests) that, enabled by their chemical structure and size, form inclusion complexes where the guest is surrounded by the macrocyclic host²⁶. Among these, CBs and CDs have been widely preferred as hosts since they exhibit good biocompatibilities in biomedical field.

While CBs can form both inclusion and exclusion complexes with various organic compounds, host-guest chemistry typically refers to the formation of inclusion complexes, where the “guest” is inserted through the macrocycle of the CB. In this case, the displacement of water molecules as well as hydrophobic interactions inside the cycle lead to particularly strong interactions and thermodynamically favorable binding. Hence, “guest” size and the presence of an appropriately sized host is a major determinant of interaction strength. The presence of tightly arranged polar, charged, and hydrogen-bonding groups on the “rim” of CB can also lead to the formation of exclusion complexes, especially if the molecule is too bulky to fit into the cavity but for organic molecules, this tends to be weaker. As such, “host-guest” interactions between CBs and organic compounds generally refer automatically to inclusion complexes, but CBs can also exhibit quite strong interactions with charged/polar groups through their rims and form exclusion complexes.

Typically, a “guest” that exhibits “host-guest interactions”, i.e., forms an inclusion complex, with CB easily displaces a compound that forms an exclusion complex. Unlike most synthetic hydrogels with permanent chemical crosslinks, SHs can undergo dissolution in response to various environmental stimuli and self-heal when damaged³⁰⁻³². SHs that rely on host-guest chemistry or the formation of exclusion complexes with a macrocyclic ligand can easily be dissembled by adding a compound that has a very high affinity to a host and can break the physical crosslinks, serving as a dissolution agent (DSA). However, while SHs display unique dynamic features, their mechanical properties are often inadequate for clinical use. Integration of benign inorganic materials (e.g., clay) or other rigid and strong nanomaterials to form supramolecular “hybrid” hydrogels (SHHs) can improve mechanical properties beyond SHs³³⁻³⁷. This approach has not yet been explored in the context of dissolvable burn dressings.

### SUMMARY OF THE DISCLOSURE

A supramolecular “hybrid” hydrogel (SHH) for treating second-degree burns is disclosed. The design goals for this SHH were a) simple fabrication, b) rapid on-demand dissolution for easy removal, c) high mechanical toughness, d) rapid, autonomous self-healing, and e) high biocompatibility in vitro and in vivo. The synthesis approach does not require any toxic compounds, catalysts, and initiators unlike the commonly used CCHs, and thus advances “green chemistry” strategies³⁸⁻⁴¹. An important feature of this invention is that the gel can be dissolved when desired using a dissolution agent (DSA), removing the need for the painful and time-consuming dressing removal processes.

Specifically, the proposed SHHs comprise three main components:
1) a water-soluble polymer that comprises functional groups capable of interacting with a macrocyclic ligand molecule. Preferred functional groups are typically cationic moieties (e.g., amine groups) that exhibit dipole-ion, dipole-dipole, and hydrogen bonding interactions with the macrocycle. This polymer can be a homopolymer or a co-polymer comprising more than one type of repeat unit.

One example of this water-soluble polymer that is included in examples and was demonstrated experimentally is a copolymer (CP) that comprises the quaternary amine containing monomer 3-[(Methacryloylamino)propyl]trimethylammonium chloride (MATMAC), with a comonomer, for example, acrylamide (Am), (Am-r-MATMAC). Another example involves a copolymer of MATMAC with an alternative acrylic monomer, methyl methacrylate. Another example of this water-soluble polymer is a homopolymer of the zwitterionic monomer methacryloxyphosphorylcholine (MPC), which is overall neutral but comprises quaternary amine sites in the zwitterionic side-groups. Finally, commercial amine-containing polymers such as poly(ethylene imine) (PEI) may also form hydrogels.
2) the macrocyclee described above, for example, cucurbit[7]uril (CB[7]) molecules,
3) A biocompatible, strong and rigid nanomaterial, for example, exfoliated clay nanosheets (CNSs),
and optionally, 4) an anionic polymeric stabilizer, for example, sodium polyacrylate (SPA, anionic) that improves the stability of the suspended nanomaterial (FIG. 1A).

The hydrogels are formed by combining an aqueous solution of the water-soluble polymer with a solution comprising the macrocycle; the nanomaterial, with or without the stabilizer, can be included in either solution or mixed in separately (FIG. 1B). The combination of these four components, i.e., the interacting polymer (e.g., a cationic copolymer, a zwitterionic monomer, an amine containing polymer), the macrocycle (e.g., a cucurbituril, specifically CB[7], and the benign nanomaterial (e.g., exfoliated clay nanosheets, with or without stabilizer), results in mechanically strong and stable supramolecular hybrid hydrogels in less than a minute. Importantly, these SHHs are also easily and rapidly dissoluble by a dissolution agent (DSA), a molecule that interacts more strongly with the macrocycle than the functional groups of the polymer and preferably forms host-guest interactions/inclusion complexes, for example, amantadine hydrochloride (AH). Specifically, fast (4-6 minutes) on-demand dissolution were achieved via the host-guest exchange mechanism upon exposure. This is radically faster than those dissoluble hydrogels using more complex synthesis methods^{11,12,18,19}. These SHHs can autonomously self-heal in one minute through the reversible host-guest interactions, which is critical for improving the lifetime and performance of hydrogels in the clinic. SHHs must have minimal to no cytotoxicity in both in vitro and in vivo experiments, crucial for clinical translation. In vitro cytotoxicity tests for one SHH formulation described here showed that SHHs and the dissolution agent amantadine hydrochloride (AH) did not have any adverse effects on human dermal fibroblasts (HDF) and human epidermal keratinocytes (HEK). Similarly, the cytokine response to these SHHs was minimal (comparable to the negative control group), indicating that the SHHs did not activate HDF and HEK in vitro. In vivo histology and immunofluorescence staining data in mice concluded that no immune and inflammatory cells populated in the subcutaneous tissue and circulating cytokines (interleukin 6 (IL-6), granulocyte colony stimulating factor (G-CSF), keratinocyte chemoattractant (KC)) in blood showed similar levels with the sham group.

The results demonstrate that SHHs prepared using this approach can be mechanically tough, rapidly on-demand dissoluble, self-healable, and biocompatible. The synthesis method has radical simplicity (two steps), speed (<1 min), and scalability during both manufacture and use, which distinguishes the approach from existing methods. Therefore, this method promises to be a straightforward, rapid, and cost-effective approach for engineering clinical dressings for burn wound healing applications.

### BRIEF DESCRIPTION OF THE DRAWINGS

FIG. 1. A) Components of a supramolecular hybrid hydrogel. 1. Am-r-MATMAC CP: Acrylamide-co-(methacryloylamino) propyl]trimethylammonium chloride copolymer; 2. CB[7]: Cucurbit[7]uril, 3&4. CNS: Clay nanosheets and SPA: Sodium polyacrylate. The cationic edges of the aggregated CNS interacted with anionic SPA; thus, CNS dispersed in water, B) Synthesis, formation and on-demand dissolution of supramolecular hybrid hydrogels.

FIG. 2. Non-covalent hydrogelation and rheological properties (20 °C) of hydrogels. A) Control 1 does not contain CP, B) Hydrogelation of control 2. Control 2 does not contain CB[7], C) Hydrogelation of SHH-4. G′ and G″ of hydrogels at weight ratios CNS/CB[7]/SPA/CP of 2/0.26/0.06/0.15 (D, SHH-1), 2/0.26/0.14/0.15 (E, SHH-3), 2/0.26/0.14/0.25 (F, SHH-4), 2/0.26/0/0.25 (G, SHH-5), 4/0.26/0.14/0.50 (H, SHH-10), and 4/0.26/0.14/0.25 (1, SHH-12) on frequency sweep from (0.1 to 80 rad/s). 3 separate batches of hydrogels were synthesized (N=3) and tested for each experiment. Data are shown as mean±SE (N=3).

FIG. 3. On-demand dissolution and self-healing of the SHH-4. A) 20 mM and B) 40 mM AH soaked gauze was administered approximately to one-third of the hydrogel. Rhodamine B (A) and methylene blue (B) dye were added to the hydrogel for visualization. Complete hydrogel dissolution was achieved in 6 and 4 min with 20 mM and 40 mM AH-soaked gauze, respectively. The thickness of SHHs: 2 mm. Scale bar: 1.5 cm. C) Two flower-shaped SHHs were cut and spliced. Hybrid hydrogels self-healed in 1 min. without any external force. The thickness of SHHs: 4 mm, Scale bar: 1.5.

FIG. 4. In vitro toxicity of dissolution agent (amantadine hydrochloride (AH)) In vitro cytotoxicity of AH solution with A) human dermal fibroblasts (HDF), B) human epidermal keratinocytes (HEK). Cells were treated with AH for 10 min (2 & 10 mM), 6 min (20 mM), and 4 min (40 mM). Culture media was used for controls. Presto blue assay of AH exposed C) HDF and D) HEK. Dermal and epidermal cells treated with 2, 10, 20 mM AH solution showed higher viability and did not differ from untreated control. Minimal adverse effect was observed with 40 mM AH. Image scale bar: 400 pm. Data are expressed as the mean±SD (n=4, N=4), *≤0.05.

FIG. 5. In vitro toxicity of supramolecular hybrid hydrogels (SHH-4). In-vitro cytotoxicity of SHH-4 with A) human dermal fibroblasts (HDF), B) human epidermal keratinocytes (HEK) after 24 hr. incubation. C) Presto blue assay of SHH-4 exposed HDF and HEK. Cells w/o SHH-4 were used for controls. SHH-4 exposed cells did not differ from untreated control. Image scale bar: 400 pm. Data are expressed as the mean±SD (n=4, N=4), *: p≤0.05, n.s: non-significant.

FIG. 6. In vitro cytokine expression of HDF and HEK after 24 h incubation with SHH-4. A) Schematic diagram of the procedure description, cytokine expression of B) HDF and C) HEK after SHH-4 exposure. LPS administration (10 ng/mL for HDF and 100 ng/mL for HEK) was used as positive controls. The HDF and HEK cultured without the gel were used as negative controls. Data are expressed as mean±SD (n=4, N=4). We use *: p≤0.05, **: p≤0.01, ***: p≤0.001 by Tukey-test for significance comparisons between cytokine levels in the untreated and SHH-4-treated groups. Cells incubated with SHH-4 did not differ from media-only controls and were significantly lower than the positive control (LPS). FIG. 6A was created with BioRender.com.

FIG. 7. In vivo biocompatibility of supramolecular hybrid hydrogel (SHH-4). A) Schematic diagram of the procedure description. B) Histology results for mice with and without SHH-4 treatment. EPI: Epidermis, M: Muscle, SISM: Small intestinal submucosa. Scale bar: 200 pm. Immunofluorescence staining of mice tissue samples with C) CD45 and D) CD68. Scale bar: 300 μm. E) in vivo circulating cytokine (blood-plasma) analysis for mice with and without SHH-4 treatment. Data are expressed as mean±SD. Sham control: A pocket was made but no gel implanted. Positive control: Small intestinal submucosa subcutaneously implanted to trigger immune response in mice. N=5 for negative control, n=5 for SHH-4 and n=4 for positive controls were used in the experiment. (IL-6: Interleukin 6, G-CSF: granulocyte colony stimulating factor, and KC: keratinocyte chemoattractant. We use *: p≤0.05, n.s.: non-significant by Tukey-test for significance comparisons in E FIG. 7A was created with BioRender.com.

FIG. 8. Synthesis scheme for A) free radical polymerization (FRP) of the random copolymer Am-r-MATMAC, and B) 1H-NMR spectrum of the Am-r-MATMAC.

FIG. 9. Interaction of CB[7] and MATMAC. 1H-NMR spectrum of A) CB[7], B) MATMAC, C) CB[7]/MATMAC ratio: 1/1, D) CB[7]/MATMAC ratio: %, E) CB[7]/MATMAC ratio: 1/3.

FIG. 10. Non-covalent hydrogelation of supramolecular hybrid hydrogels. 3 separate batches of hydrogels were synthesized (N=3) for each experiment.

FIG. 11. Rheological properties (20 °C) of hydrogels with different SPA, CP, CB[7] and CNS concentrations. Storage (G′) and loss (G″) modulus of hydrogels at weight ratios CNS/CB[7]/SPA/CP of 2/0.26/0.1/0.15 (A, SHH-2), 2/0.26/0.14/0.50 (B, SHH-6), 2/0.52/0.14/0.50 (C, SHH-7), 2.5/0.26/0.14/0.25 (D, SHH-8), 3/0.26/0.14/0.50 (E, SHH-9), and 3/0.26/0.14/0.25 (F, SHH-11) on frequency sweep from (0.1 to 80 rad/s). 3 separate batches of hydrogels were synthesized (N=3) and tested for each experiment. The data are expressed as mean±SE (N=3).

FIG. 12. Comparison of the rheological properties of SHH-4 with and without CB[7](Control 2 at weight ratios of CNS/CB[7]/SPA/CP of 2/0/0.14/0.25, SHH-4 at weight ratios of CNS/CB[7]/SPA/CP of 2/0.26/0.14/0.25). Storage (G′) and loss (G″) modulus of A) Control 2 (SHH-4 w/o CB[7]), and B) SHH-4 on frequency sweep from (0.1 to 80 rad/s). G′ and G″ of C) Control 2 (SHH-4 w/o CB[7]), D) SHH-4 in oscillatory strain sweep measurements. The data are expressed as mean±SE (N=3).

FIG. 13. Statistical analyses of storage modulus (G′) values of SHHs. Data are expressed as mean±SE (N=3). We use *: p≤0.05, **: p≤0.01, ***: p≤0.001 by Tukey-test for significance comparisons between G′ among different groups.

FIG. 14. On-demand dissolution of SHH-4 without CB[7]. A) 20 mM and B) 40 mM AH soaked gauze was administered approximately to one-third of the SHH-4 without CB[7]. Rhodamine B (A) and methylene blue (B) dye were added to the hydrogel for visualization. No dissolution was achieved in 6 hours with 20 mM and 40 mM AH-soaked gauze. The thickness of SHH-4 w/o CB[7]: 2 mm. Scale bar: 1.5 cm. 3 separate batches of hydrogels were synthesized (N=3) for each experiment.

FIG. 15. Self-healing ability (20 °C) of SHH-4. Step-rate time-sweep measurements to affirm the self-healing ability of SHH-4. This continuous measurement was repeated 3 times. The data are expressed as mean±SE (N=3).

FIG. 16. A) Effects of pH on SHH-4 stability, B) swelling ratio of SHH-4 at different time intervals and pH values. The data are expressed as mean±standard deviation (N=3).

FIG. 17. In vitro toxicity of dissolution agent (amantadine hydrochloride (AH), memantine hydrochloride (MH)) after treatment with HDF and HEK. A) and B) 2, 10, 60 mM AH treatment of HDF and HEK. We treated cells with AH for 10 min (2 & 10 mM), and 4 min (60 mM). We used culture media for controls. No adverse effects were observed after 2 and 10 mM AH treatment. However, 60 mM AH detached HDF and HEK from the surface of the wells. C) and D) 2, 10, and 20 mM MH treatment of HDF and HEK. E) and F) We treated cells with MH for 10 min (2 & 10 mM), and 6 min (20 mM). We used culture media for controls. No adverse effect was observed with 2 and 10 mM MH concentration. However, 20 mM MH detached some of the HDF and HEK from the surface of the wells. Image scale bar: 400 μm. The data are shown as the mean±SD (n=4, N=4), *<0.05.

FIG. 18. SHH dressings for on-demand dissolution and easy burn care.

FIG. 19. ¹H-NMR spectrum of poly (methyl methacrylate-random-3-(methacryloylamino) propyl]trimethylammonium chloride) (P-MMA-r-MATMAC).

FIG. 20. ¹H-NMR spectrum of poly (2-methacryloyloxyethyl phosphorylcholine).

FIG. 21. Interactions between CB[7] and MPC. 1H-NMR spectrum of Top) CB[7], Middle) MPC, Bottom) CB[7]/MPC molar ratio: 1/1.

FIG. 22A-D. Hydrogel dissolution using PBS 1×.

### DETAILED DESCRIPTION

Supramolecular “hybrid” hydrogels (SHHs) that are the focus of this invention can be formed by combining the following components:
1. A water-soluble polymer that comprises functional groups that are capable of interacting with a macrocyclic ligand molecule.
2. The macrocyclic ligand molecule (described above)
3. A biocompatible nanomaterial

In addition, an optional additional water-soluble polymer that stabilizes the suspension of the nanomaterial may be used.

An important feature of these SHHs is that they can be dissolved on demand by exposing the hydrogel to a small molecule that interacts more strongly with the macrocyclic molecule than the polymer, termed a dissolution agent (DSA). The DSA is typically a molecule that exhibits strong host-guest interactions with the macrocycle, displacing the polymer side-groups and breaking supramolecular cross-links.

The water-soluble polymer can be a homopolymer with only one type of repeat unit, or a copolymer (CP) that comprises more than one type of repeat unit.

The macrocyclic molecule can be selected among cucurbiturils, rotaxanes, cyclodextrins, and cyclic peptides. Preferred embodiments use cucurbiturils and cucurbituril derivatives with at least some water solubility. Among the cucurbituril family, CB[7] was selected for this proof of concept since it has particularly high water solubility, up to 30 mM²⁷, and low cytotoxicity⁴⁵. The portals of CB[7]s are highly electronegative and make them highly attractive for cation binding through the ion-dipole effect⁴⁶ and enables it to form exclusion complexes.

The water-soluble polymer must have side-groups that exhibit strong interactions with the macrocyclic group. These interactions may be host-guest interactions, where the interacting side-group forms an inclusion complex with the macrocycle where the side-group enters the cavity of the macrocycle. Alternatively, these interactions may involve the formation of and exclusion complex, where the side-groups of the polymer interact with the “rims” of the macrocycle through a combination of electrostatic, ion-dipole, dipole-dipole, and hydrogen-bonding interactions.

The biocompatible nanomaterial has to have sufficient strength, rigidity, and dispersability to create good hybrid hydrogels with improved strength. This initial study shows data with exfoliated clay nanosheets (CNSs)^{33-37,43,44}, with or without the addition of sodium polyacrylate (SPA) as a stabilizer^{33,35-37,43,44}. Other nanomaterials include nanocellulose-based materials (e.g., nanowhiskers, nanofibers, nanocrystals, bacterial nanocellulose), chitosan- and chitin-based nanomaterials, starch-based nanomaterials, ceramic nanoparticles (e.g., silica, mesoporous silica, hydroxyapatite, aluminum oxide, zinc oxide, iron oxide, titanium dioxide), metallic nanoparticles (e.g., silver, gold), graphene oxide, and carbon nanotubes.

In one embodiment, the water-soluble polymer was a custom-designed water-soluble copolymer (CPs), for example, 3-[(Methacryloylamino)propyl]trimethylammonium chloride (MATMAC) with a comonomer, for example, acrylamide (Am), (Am-r-MATMAC) CP.

The SHHs using this polymer were synthesized simply by mixing two aqueous solutions, one containing, for example, the Am-r-MATMAC CP and the other containing, for example, CB[7] and CNS with or without SPA.

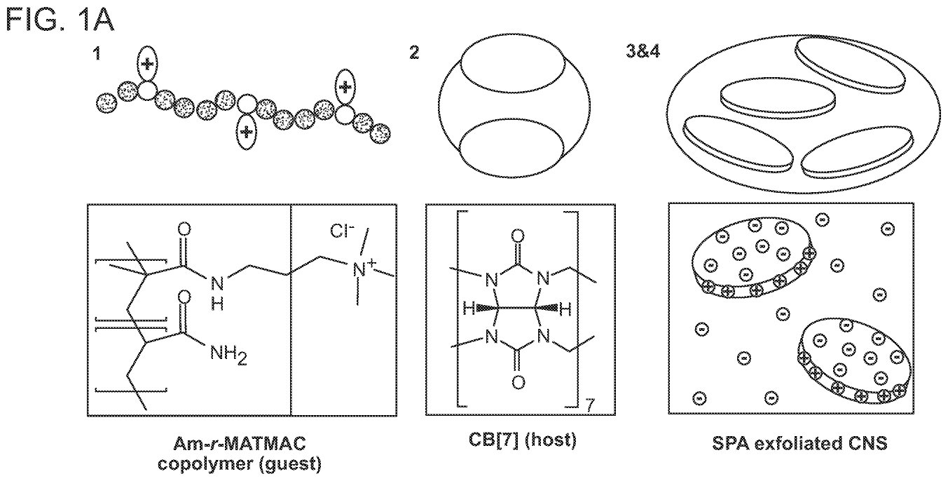

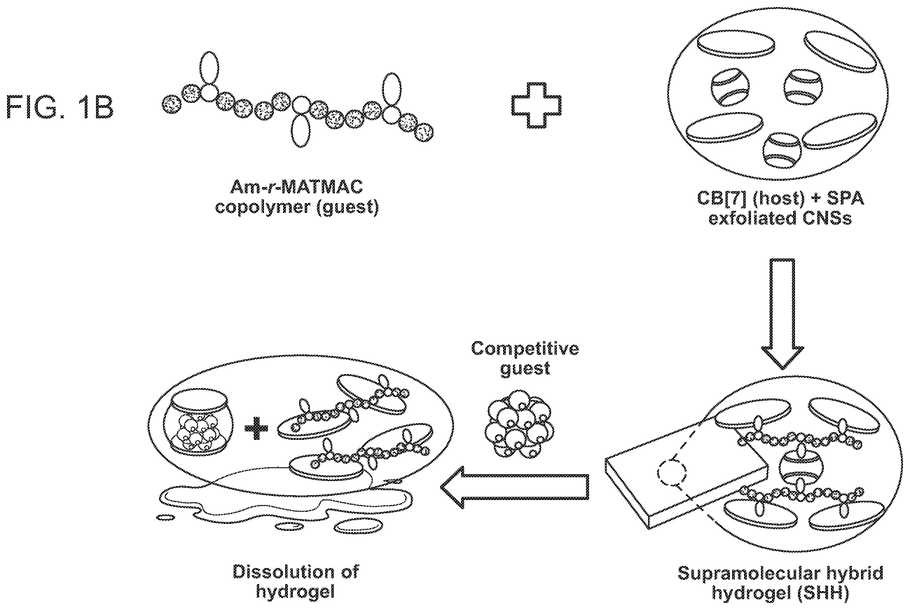

For the dissolution of the SHH, a solution containing a dissolution agent (DSA) is applied to the hydrogel. The DSA is a molecule that binds the macrocycle (in this example CB[7]) more strongly than the functional groups on the polymer. The DSA can be chosen among solutes with very high affinities to CB[7]. Typically, the DSA forms inclusion complexes with the macrocycle, displacing the polymer. An example is amantadine hydrochloride (AH). When added to the medium, the DSA molecules displace and replace the quaternary amine groups of the CP interacting with CB[7], breaking these physical crosslinks. This results in the dissolution of the SHH (FIG. 1B).

A simple, quick, and scalable method to synthesize a supramolecular hybrid hydrogel (SHH) via the supramolecular assembly of Am-r-MATMAC CP with CB[7] ligand s and CNSs was discovered. The SHH design and synthesis feature a green chemistry—whereby many toxic and reactive chemicals commonly used traditional approaches were eliminated—which also results in rapid hydrogel formation (~15 seconds to gelation). As such, these SHHs are the first of their kind as burn dressings and drastically differ from the existing lengthy and potentially hazardous methods of hydrogel preparation. Furthermore, the non-covalent exchange mechanism between the Am-r-MATMAC CP and CB[7], in the SHHs, enables rapid on-demand dissolution (4-6 min) of the hybrid hydrogels. This dissolution is facilitated by the introduction of a dissolution agent, AH (20 & 40 mM), without any significant adverse effects on either dermal and or epidermal cells in vitro (cell viability >90%) and in-vivo.

Supramolecular crosslinks with CNSs provided distinctive functions of high mechanical strength (G′>50 kPa; G′>G″) and fast self-healing capability (1 min) for SHH-4. In addition, SHH-4 showed high biocompatibility in vitro and in vivo. In vitro immunogenicity assessment showed lower immune response to SHHs compared to positive (LPS) controls. Moreover, after subcutaneous hydrogel implantation in mice, in vivo histology, immunofluorescence and systemic cytokine analyses indicated no immune and inflammatory responses beyond sham controls. As such, SHH-4 is a promising alternative to commercial hydrogels currently used in clinical burn wound treatment.

Supramolecular crosslinks with CNSs provide distinctive functions of high mechanical strength and fast self-healing capability. For example, one SHH sample, SHH-4, showed better mechanical strength than other supramolecular hydrogels in the literature (G′>50 kPa; G′>G″), exhibited self-healing in ~1 minute, and showed high biocompatibility in vitro and in vivo. In vitro immunogenicity assessment showed lower immune response to SHHs compared to positive (LPS) controls. Moreover, after subcutaneous hydrogel implantation in mice, in vivo histology, immunofluorescence and systemic cytokine analyses indicated no immune and inflammatory responses beyond sham controls. As such, SHH-4 is a promising alternative to commercial hydrogels currently used in clinical burn wound treatment.

In another embodiment, the water-soluble polymer combines the same cationic, interacting MATMAC monomer with a more hydrophobic monomer such as methyl methacrylate (MMA). This copolymer, poly (methyl methacrylate-random-3-(methacryloylamino) propyl]trimethylammonium chloride) (P-MMA-r-MATMAC, abbreviated as PMM), can also form strong SHHs (G′>20 kPa) that are dissoluble. Being able to tune the hydrophobicity of the copolymer is important for controlling water uptake, swellability, and stability of SHHs.

In another embodiment, the water-soluble polymer may be zwitterionic, i.e., with an overall neutral charge but synthesized from monomers that each have both an anionic and a cationic group. An example of such a polymer is poly(methacryloxy phosphorylcholine) (PMPC). The zwitterionic phosphorylcholine groups combine an anionic phosphate moiety with the cationic quaternary amine moiety at the end of the side-group. This polymer is reported to be hemocompatible, biocompatible, and superhydrophilic, all highly desired properties for wound and burn dressings. MPC side-groups exhibit interactions with CB[7], likely forming exclusion complexes. NMR analysis shows that, when MPC is mixed with CB[7], peaks associated with the methyl groups on the quaternary amine moiety shift significantly, indicating shielding due to interaction with the macrocycle. PMPC-based SHHs can also exhibit excellent mechanical properties (G′>20 kPa) along with dissolubility.

Even commercial amine-containing polymers such as poly(ethylene imine) (PEI) can form SHHs using the same procedure. However, its mechanical properties and dissolubility is not as favorable as the polymers described above.

Preferred polymer side-groups appear to involve a quaternary amine group, whether resulting in a cationic repeat unit (e.g., MATMAC) or as part of a zwitterionic group (MPC). Various other repeat units that incorporate quaternary amine groups are likely good choices. While the presence of alkyl groups surrounding the cationic site (such as the methyl groups on the quaternary amines here) can prevent or limit the formation of inclusion complexes with cucurbiturils, in this case they appear to form strong supramolecular interactions nonetheless.

Other potential functional groups that may be promising include tertiary amines, secondary amines, imidazolium and pyridinium groups, etc.

Dynamic Mechanical Analysis (DMA) can be used to measure the storage modulus (G′) (describes elasticity) and loss modulus (G″) (describes viscosity) versus frequency (ω=0.06-60 rads-1). In some embodiments, the G′ of SHHs is larger than G″ over a wide frequency range, signifying a stable hydrogel. Elastic recovery of SHHs was studied by strain amplitude sweep tests and continuous step strain measurements. Tensile tests can be conducted to determine Young's modulus, strain at break, and tensile strength.

Fluid absorption capacity: Hydrogels absorb and hold the wound exudate, which facilitates proliferation of fibroblasts and the migration of keratinocytes, both essential for the complete epithelialization of the wound. As hydrogels swell they trap wound debris and bacteria in the gel matrix, potentially reducing wound bioburden. In some embodiments the SHHs may achieve up to 500% swelling. The pH of blister fluid changes between 5-10 in second degree burns. To mimic it, we utilize acidic, neutral and basic buffer solutions including acetate buffer (pH 5), phosphate buffer (pH 5.8-8)) and glycine-sodium hydroxide buffer (pH 10). After freeze-drying of disk-shaped SHHs, they are weighed and then immersed in a swelling medium. Every 10 minutes, the SHH is removed from the solution and weighed after excess solution on the surface is blotted55 until the gel is saturated and a stable mass is reached. Swelling results are assessed using the following equation: Q=(Ws−Wd)/Wd, where Q is the swelling ratio, Ws is the mass in the swollen state, and Wd is the mass in the dried state.

On-demand dissolution: In some embodiments, the SHHs dissolve upon exposure to dissolution agents bind CB[7] more strongly than the cationic groups on the water-soluble polymer, displacing it from CB[7] and breaking supramolecular cross-links. The dissolution agent will also shield electrostatic interactions between the CP and the clay nanosheets. In some embodiments, the hydrogel achieves on-demand dissolution (<5 minutes).

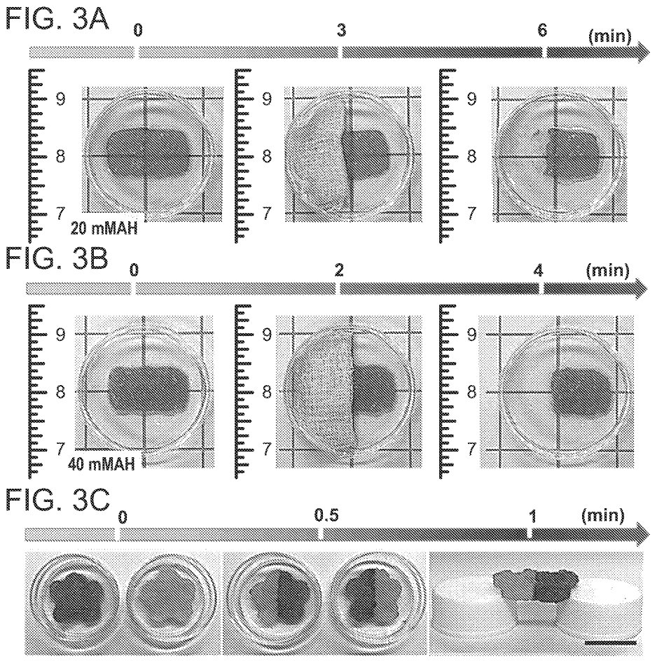

Aqueous dissolution agent (DSA) solutions with different concentrations (0-100 mM) of the dissolution agent were prepared. SHHs of various thicknesses of (2×1 cm2, 1-5 mm) were used to observe dissolution kinetics. A gauze soaked in the DSA solution (0-100 mM) was placed on SHH pieces such that the gauze covers ~50% of the SHH surface area (FIG. 3A & B). New DSA-soaked gauze pieces was introduced until complete hydrogel dissolution was achieved. In some embodiments, faster dissolution kinetics are obtained at high concentrations of DSAs and low thicknesses. While higher SPA, CNS and water-soluble polymer contents may improve mechanical strength, they can also slow down dissolution kinetics and prolong on-demand dissolution time.

Self-healing capability: Self-healing hydrogels can spontaneously/autonomously repair their damage and recover performance upon unexpected wear and tear while in use; thus, improving the useful lifetime and performance of hydrogels. Non-covalent interactions in SHHs allow quick and continuous recovery of cross-links and thus eliminate the need for reactive chemicals or toxic catalysts for self-healing in CCHs. We assessed the self-healing of SHHs. We prepared two SHHs soaked in methylene blue and rhodamine B for visibility. FIG. 3C. SHHs are cut into two pieces with a razor, then spliced together and allowed to self-heal. The strength of the resulting block was assessed by suspending horizontally and holding vertically with a needle. In some embodiments, full self-healing is complete in 1-2 min, similar to the current state-of-the art.

In some embodiments, the SHH dressings have good and stable mechanical properties (G′>1 kPa; G′>G″), rapid on-demand (≤5 min) dissolution behavior, and self-healing capability (1-2 min).

Due to the ease of their fabrication, which only involves two steps of mixing, these novel SHHs will enable large-scale yet low-cost fabrication, addressing a critical bottleneck for translation to clinical applications. Further, due to this ease and the speed (15 seconds) of SHH formation, the dressings could be formed at the bedside and on-field synthesis of the patients with customizations as necessary. Such novel and on-demand dissoluble SHHs have great potential as second-degree burn dressings. SHH-based dressings provide easy burn care, eliminate mechanical and surgical debridement, promote wound healing and enhance the healing process to treat second-degree burns. As a result, SHHs will reduce 1) pain and psychological burden of patients, 2) use and side effects of heavily used analgesics and opioids, 3) hospital stay and costs, and 4) time demand on highly qualified hospital personnel (i.e, doctors and nurse practitioners) by reducing the dressing change time significantly. To this end, we will follow up this study with investigations on the in vivo efficacy of SHHs in burn wound healing progress in large animal model with further SHH optimizations—via perturbations to the composition of existing formulations—and clinical trials to show the translatability of novel SHHs to the clinic. The invention removes the challenges and shortcomings associated with current burn dressings and bear reduced pain, easy to apply, and removable burn dressings.

SHHs find many further uses in the broader areas of biomedical engineering, such as tissue and micro-tissue engineering, regenerative medicine, and biopreservation. SHHs can be further decorated with functional surface groups as needed for such purposes. The quick dissolubility of the SHHs is especially desirable where sacrificial materials are useful for fabrication and/or as temporary closure elements. The tunable properties of SHHs lends itself to cell and 3D tissue culture where the specimens can first be fixed in space for imaging and observation in the SHHs but then released for detailed molecular analysis through the dissolution process in under a minute whereas alternatives take much longer. Such engineered tissue constructs are also useful both in the biopreservation of tissues and subsequent transplant applications.

### EXAMPLES

### SHH Synthesis and Characterization

Chemicals: Acrylamide (Am), [3-(Methacryloylamino) propyl]trimethylammonium chloride solution, 50 wt. % in H₂O (MATMAC), cucurbit[7]uril hydrate (CB[7]), sodium polyacrylate (SPA), amantadine hydrochloride (AH), memantine hydrochloride (MH), and N, N, N′, N′-Tetramethyl ethylenediamine (TEMED), lipopolysaccharides from Escherichia coli O111:B4-LPS (LPSs), citrate buffer (pH 6), Tris-EDTA buffer (pH 8) were all purchased from Sigma Aldrich (St. Louis, MO). Laponite XLG-XR (clay nanosheets) was acquired from Acme-Hardesty Co. (Blue Pell, PA). Sodium bicarbonate buffer (pH 10) was purchased from Bioworld (Dublin, OH). Acetate buffer, reagent alcohol, acetonitrile (ACN) and ammonium persulfate (AMPS), Live/Dead Cell Viability/Cytotoxicity Kit, and presto blue were obtained from Thermo Fisher Scientific (Waltham, MA). Deuterated methanol was sourced from Cambridge Isotope Laboratories Inc. (Tewksbury, MA). Hematoxylin and eosin were purchased from Leica Biosystems (Germany). All chemicals and solvents were of reagent grade and used as received.

### Example 1

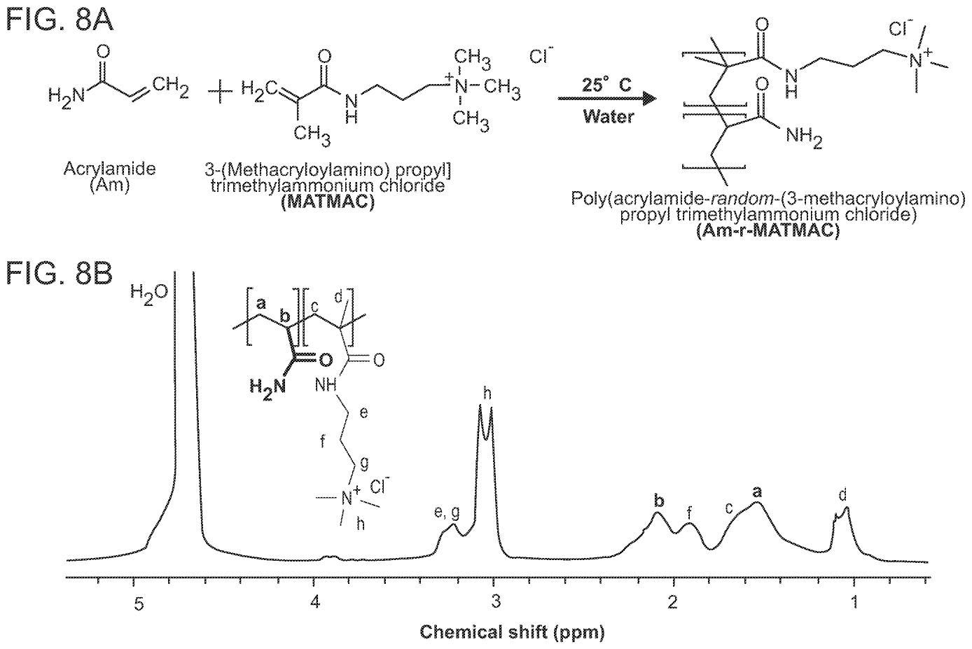

Synthesis of Poly (acrylamide-random-3-(methacryloylamino)propyl] trimethylammonium chloride (P-Am-r-MATMAC)—Abbreviated as PAM: The random copolymer poly(acrylamide-random-(3-methacryloylamino) propyl trimethylammonium chloride) (Am-r-MATMAC) was synthesized following a free radical polymerization (FIG. 8A). Briefly, 6 mL MATMAC and 7 g Am were dissolved in 100 mL distilled water in a 250 mL round bottom flask at room temperature. 0.1 g of the initiator AMPS was added after dissolution. The reaction mixture was purged with nitrogen for 15 min. Then, 0.067 mL TEMED in 0.5 mL water was added into the mixture dropwise with a syringe. After 15 min nitrogen purge, the reaction was conducted by stirring at 250 rpm for an hour at room temperature (25 °C). Then, the reaction mixture was poured into a 1:1 mixture of ACN and reagent alcohol to precipitate out the copolymer and followed by three successive washes to eliminate any remaining unreacted monomer. The attained solid polymer was dried for two days under a fume hood and two more days in a vacuum oven at 50 °C. The product yield was 75% and calculated from the ratio of the mass of the product copolymer to the mass of the monomers used. The chemical composition of the copolymer was obtained by ¹H NMR (Bruker Avance III 500 MHz spectrometer, DMSO-d6). The calculated composition was: 46 wt % MATMAC, and 54 wt % Am. The binding interaction between the ligand (CB[7]) and the water-soluble polymer (Am-r-MATMAC) was also confirmed by ¹H NMR in a neutral D₂O solution using different CB[7] and MATMAC ratios.

Synthesis of SHHs: For SHH-4 synthesis, SPA (4.2 mg) was added to a stirred suspension of CNSs (60 mg) in water (3 mL). After 15 minutes, the addition of SPA stabilized the exfoliation of CNSs. Then, CB[7] (8 mg) was added to this mixture, and the mixture was vortexed to achieve complete dissolution. Finally, Am-r-MATMAC copolymer (CP) (7.3 mg) was added, and the mixture became completely stiff within only 15 seconds, forming a hydrogel. As a control, we performed the same protocol with only SPA (1.8 mg), CNSs (60 mg) and CB[7] (8 mg) and without the Am-r-MATMAC copolymer. Different concentrations of CNS, CB[7], SPA, CP, were also tested to assess hydrogels' mechanical properties. Selected hydrogel (SHH-4) was tested for dissolution kinetics (Table 1).

Rheological Tests: The rheological properties of SHHs were measured at 25 °C using a rheometer (TA Instruments, Discovery HR 20 Rheometer, New Castle, DE) fitted with parallel plates (diameter 20 mm). The gap at the apex of the para-plate was set to be 2 mm. The samples (8 mm dia.≈2 mm thickness) were placed between the para-plate and the platform with special care to avoid water evaporation. Dynamic frequency sweep tests (0.1-80 rad/s) at a fixed strain (γ) of 0.5% were measured to determine the storage modulus (G′) and loss modulus (G″) of hydrogels. Three different batches of SHHs (N=3) were tested for rheological tests. To investigate the self-healing properties of hydrogels, oscillatory strain sweep measurements were performed (frequency, ω=6.0 rad/s (1.0 Hz)) to determine the collapse of the SHH from gel state to a quasi-liquid state. Then, step-rate time-sweep measurements were performed using the following procedure: frequency, ω=6.0 rad/s (1.0 Hz), SHH-4 was subjected to 0.1% strain for 300 s, then 600% strain was applied to damage the hydrogel for 150 s and later strain went back to 0.1% for recovery for another 300 s. This continuous measurement was repeated 3 times. Three different batches of SHHs (N=3) were tested for each rheological test.

Compression tests: Mechanical properties (Young's modulus) were analyzed using TA Instruments RSAIII Dynamic Mechanical Analyzer (DMA), (Rheometrics Solids Analyzer). SHHs, 8 mm in diameter, were prepared for compression testing. The linear behavior in the stress-strain curve at strain levels below 1% allowed using Hooke's law (E=σ/ε, where σ is the applied stress and ε is the resultant strain) to calculate Young's modulus.

On-demand dissolution: We prepared different concentrations of high binding constant dissolution agent (20, 40 mM) to observe the dissolution kinetics. Amantadine hydrochloride (AH) was used as the dissolution agent. i.e., the dissolution agent (DSA). In addition, SHHs (2×1 cm², 2 mm thickness) were prepared, and rhodamine B and methylene blue dye was added during gelation for visualization. Finally, gauze soaked in different aqueous concentrations of selected DSA solutions (20 mM AH (0.075 g) and 40 mM AH (0.15 g)) in culture media (20 mL) was administered to the hydrogel (SHH-4), and time was recorded until complete dissolution was achieved.

Self-healing ability: Two SHHs (SHH-4) were prepared in a flower-shaped mold and stained with either rhodamine B (pink) or methylene blue. Then, they were cut into equal two parts. Two halves of alternate-colored hydrogels were combined to form color blended (pink and blue) flower-shaped SHHs at 25 °C without any external intervention. Self-healing was affirmed by the ability of the repaired flower-shaped hydrogel to retain its structure when hanging under gravity. Three different batches of SHHs (N=3) were tested for self-healing experiments.

pH stability and swelling ratio: We prepared SHHs (SHH-4) to test the pH stability and the swelling ability of hybrid hydrogels. For pH stability, SHHs were synthesized in glass bottles. After gelation was completed, 5 mL of acetate buffer (pH 5), citrate buffer (pH 6), PBS buffer (pH 7.4), tris-EDTA buffer (pH 8), sodium bicarbonate buffer (pH 10, 1M) was added into each bottle, and SHHs allowed to stand at room temperature for 72 hrs.

Swelling measurements were performed gravimetrically. The SHHs (SHH-4, 4 mm dia., 4 mm thickness) were dried in an oven at 60 °C for an hour. Then dried SHHs with known weights were immersed in the PBS at 37 °C. The swollen samples were taken out and weighted at predetermined time intervals. The swelling ratio was determined using the following equation:

$$Swelling ratio ( \% ) = {W}_{s} / {W}_{i} * 100 ,$$

wherein Wₛ and Wᵢ were the weights of hydrogels at the swelling state and the initial state, respectively¹⁸.

Three different batches of SHHs (N=3) were tested both pH stability and swelling measurements.

### In Vitro Biocompatibility of SHHs

Cell lines and Culture: Cryopreserved human dermal fibroblasts (HDF) were obtained from Cell Applications (San Diego, CA) and culture in fibroblast growth medium (Sigma Aldrich (St. Louis, MO)). Primary human epidermal keratinocytes (HEK), keratinocyte growth kit, dermal cell basal medium, and phenol red were purchased from ATCC (Manassas, VA). Epidermal cells' media were supplemented with 1% penicillin-streptomycin solution (ATCC, Manassas, VA). HDF and HEK were cultured and passaged according to the protocols provided by the manufacturer.

SHHs toxicity: HDF and HEK were seeded into 24 well plates with an initial density of 10000 cells per well. The cells were incubated for 48 h in their respective culture media to allow attachment and reach confluency before the toxicity experiments. Then, the media in each well was removed, and the cells were rinsed with PBS. SHHs (8 mm dia.≈1 mm thickness) were suspended in sterile PBS for two days and then were added into the wells where they were incubated with the different cells (n=4, for each group) for another 24 h. Control groups were incubated without SHHs. The cell viability was assessed by a) live/dead cell viability/cytotoxicity staining kit, and b) by presto blue assay to quantify the percentage of living cells compared to the controls. For staining, 1:250 calcein AM and 1:25 ethidium homodimer-1 combined and vortexed in PBS and 0.5 mL solution/well were added, and cells were incubated for 20 minutes at 37 °C. After PBS rinsing, the fluorescent images were captured using the Evos FL imaging system (Thermo Fisher Scientific, Waltham, MA). For quantitative analysis, presto blue was added (10% v/v) directly into cell culture media in each well (n=4, for each group) with and without SHHs hydrogel. After an hour of incubation, 100 μL of culture supernatants were collected, and the absorbance was measured in a plate reader at 570 nm and 600 nm. The percentage of living cells was calculated to controls.

Dissolution agent toxicity: HDF and HEK were cultured in 96 well plates with an initial density of 1000 cells per well. After rinsing the cells with PBS, 0.1 mL of amantadine hydrochloride (AH) (2 (0.0075 g), 10 (0.0375 g), 20 (0.0750 g), 40 (0.15 g) mM in 20 mL cell culture media) and memantine hydrochloride (MH) (2 (0.0071 g), 10 (0.035 g), 20 (0.071 g) mM) solution were added into each well. After AH and MH treatment (6 min with 2, 10, 20 mM and 4 min with 40 mM and 60 mM), live/dead cell viability staining and presto blue assay were performed as described above.

Quantification of cytokines: We collected culture supernatants of HDF and HEK after exposing cells to SHHs for 24 hrs. Cell culture and LPS supplemented media (10 ng/mL for HDF 100 ng/mL for HEK) were utilized as negative and positive controls, respectively (n=3 for each positive and negative controls, n=4 for SHHs treated groups). The levels of cytokines, chemokines, and growth factors were quantified using a Human High Sensitivity T-Cell Discovery Array 48-plex (HDHSTC48) (Eve Technologies, Calgary, AB, Canada). The multiplexing analysis was performed using the Luminex™ 200 system (Luminex. Austin, TX, USA). Forty-eight markers were simultaneously measured in the samples using Human Cytokine 48-Plex Discovery Assay® (MilliporeSigma, Burlington, Massachusetts, USA) according to the manufacturer's protocol. The 48-plex consisted of soluble CD40 ligand (sCD40L), epidermal growth factor (EGF), Eotaxin, fibroblast growth factor (FGF-2), Fms-like tyrosine kinase receptor 3 ligand (FLT-3 ligand), fractalkine, granulocyte colony stimulating factor (G-CSF), granulocyte macrophage-colony stimulating factor (GM-CSF), growth-regulated oncogene α (GROα), interferon alpha-2 (IFN-α2), interferon gamma (IFN-γ), interleukin 1 alpha (IL-1α), interleukin 1 beta (IL-1β), interleukin IRA (IL-IRA), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 12 p40 (IL-12 (p40)), interleukin 12 p70 (IL-12(p70)), interleukin 13 (IL-13), interleukin 15 (IL-15), interleukin 17A (IL-17A), interleukin 17E (IL-17E)/interleukin 17F (IL-17F), interleukin 25 (IL-25), interleukin 18 (IL-18), interleukin 22 (IL-22), interleukin 27 (IL-27), interferon gamma-induced protein 10 (IP-10), monocyte chemoattractant protein-1 (MCP-1), monocyte chemoattractant protein-3 (MCP-3), macrophage colony-stimulating factor (M-CSF), macrophage-derived chemokine (MDC), monokine induced by gamma interferon/Chemokine (C—X—C motif) ligand 9 (MIG/CXCL9), macrophage inflammatory protein-1 alpha (MIP-1α), macrophage inflammatory protein-1 beta (MIP-1β), platelet-derived growth factor AA (PDGF-AA), PDGF-AB/BB, regulated upon Activation, Normal T Cell Expressed and Presumably Secreted (RANTES), Transforming growth factor α (TGF-α), tumor necrosis factor-alpha (TNF-α), tumor necrosis factor-beta (TNF-β), vascular endothelial growth factor (VEGF-A). Assay sensitivities of these markers range from 0.14-50.78 pg/mL for the 48-plex. Individual analyte sensitivity values are available in the MILLIPLEX protocol.

### In Vivo Biocompatibility of SHHs

BALB/C mice aged 7-8 weeks, weighing 17-21 g, were used for the in-vivo biocompatibility test. The animals were supplied from Charles River Laboratories (Wilmington, MA, USA) and housed at the Center of Comparative Medicine of Massachusetts General Hospital (MGH) with access to food and water at the libitum and subjected to a 12-hr light/dark cycle at room temperature (21 °C) and relative humidity of 30-70%. All protocols were in accord with the guidelines set by the Committee on Laboratory Resources, National Institutes of Health, and reviewed and approved by the Institutional Animal Care and Use Committee (IACUC, Protocol #2015N000073) of MGH. Groups of mice (n=3-4) were used to evaluate biocompatibility. SHHs (8 mm diameter, 2 mm thickness) were immersed and sterilized in isopropyl alcohol and then PBS (pH 7.4) for 24 hr. before being subcutaneously implanted on the dorsum. As positive controls, small intestinal submucosa was used to trigger an immune response in mice⁴². As sham controls, subcutaneous pockets without SHHs were made on the dorsum. The mice were sacrificed two days after the implantation. The surrounding tissue of the implantation sites was collected and fixed for histology or homogenized for cytokine measurements. The circulating blood was drawn from the inferior vena cava for systemic cytokine measurements.

Histology Analysis: The dorsal tissues—negative, positive and SHHs implanted samples—were immersed in 10% formalin for 24-48 h at room temperature and then embedded in paraffin and cut into 5-pm sections. The sections were stained with hematoxylin and eosin (H&E) to visualize the infiltrating immune cells surrounding the biomaterial. The stained sections were imaged using a Nikon Eclipse E800 microscope (Nikon, Melville, NY).

Immunohistochemistry: Paraffin sections were deparaffinized in xylene and rehydrated in 100%, 95%, and 70% ethanol. Antigen retrieval was performed using the Declokar chamber (Biocare Medical, Concord, CA) in Declokar buffer according to the manufacturer's instructions. The sections were then blocked with 10% goat serum, followed by incubation with primary antibodies targeting CD45 (1:40, ab10558, Abcam) or CD68 (1:100, ab125212, Abcam) at 4 °C overnight. The sections were then incubated with Alexa Flour 555 secondary antibody (1:200, ab150078, Abcam). Finally, the sections were cover slipped using an antifade mounting medium containing 4, 6-diamidino-2-phenylindole (DAPI, H-180W, Vector Laboratories, Burlingame, CA). Images were taken and quantitated using the EVOS M5000 imaging system (Invitrogen, Bothell, WA).

Quantification of systemic cytokines: For systemic cytokines analysis, plasma-heparin was separated from blood by centrifugation. We used Luminex xMAP technology for multiplexed quantification of 32 Mouse cytokines, chemokines, and growth factors. The multiplexing analysis was performed using the Luminex™ 200 system (Luminex, Austin, TX, USA) by Eve Technologies Corp. (Calgary, Alberta). Thirty-two markers were simultaneously measured in the samples using Eve Technologies' Mouse Cytokine 32-Plex Discovery Assay® (Millipore Sigma, Burlington, Massachusetts, USA) according to the manufacturer's protocol. The 32-plex consisted of Eotaxin, granulocyte colony stimulating factor (G-CSF), granulocyte macrophage-colony stimulating factor (GM-CSF), interferon gamma (IFNγ), interleukin 1 alpha (IL-1α), interleukin 1 beta (IL-1β), interleukin 2 (IL-2), interleukin 3 (IL-3), interleukin 4 (IL-4), interleukin 5 (IL-5), interleukin 6 (IL-6), interleukin 7 (IL-7), interleukin 9 (IL-9), interleukin 10 (IL-10), interleukin 12 (IL-12 (p40)), interleukin 12 (IL-12 (p70)), interleukin 13 (IL-13), interleukin (IL-15), interleukin 17 (IL-17), interferon gamma-induced protein 10 (IP-10), keratinocytes-derived chemokine (KC), leukemia inhibitory factor (LIF), LPS-induced CXC chemokine (LIX), monocyte chemoattractant protein-1 (MCP-1), macrophage colony-stimulating factor (M-CSF), monokine induced by gamma interferon (MIG), macrophage inflammatory protein-1 alpha (MIP-1α), macrophage inflammatory protein-1 beta (MIP-1β), macrophage inflammatory protein-2 (MIP-2), regulated upon activation, normal T cell expressed and presumably secreted (RANTES), tumor necrosis factor (TNF-α), and vascular endothelial growth factor (VEGF). Assay sensitivities of these markers range from 0.3-30.6 pg/mL for the 32-plex. Individual analyte sensitivity values are available in the Millipore Sigma MILLIPLEX® MAP protocol.

### Statistical Analysis

All quantitative data was analyzed using the Origin Pro 2021 Graphing and Analysis Software v.9.0.8.200 (OriginLab, Northampton, Massachusetts), and are presented as the mean±standard error of the mean (SEM) from at least three hydrogels from different batches. All experiments using HDF and HEK were performed four times (experimental replicates, N=4) and at least assessed four different wells (technical replicates, n=4). To evaluate in vivo biocompatibility, totally 14 mice were used in histology, immunostaining and circulating cytokine analyses (n=4 for positive controls, n=5 for negative (sham) controls and n=5 for SHH-4 implantation). The statistical significance of the results was assessed using one-way ANOVA. Statistical significance is defined p<0.05 for all experiments.

### Characterization of SHHs

### Hydrogelation and Mechanical Properties

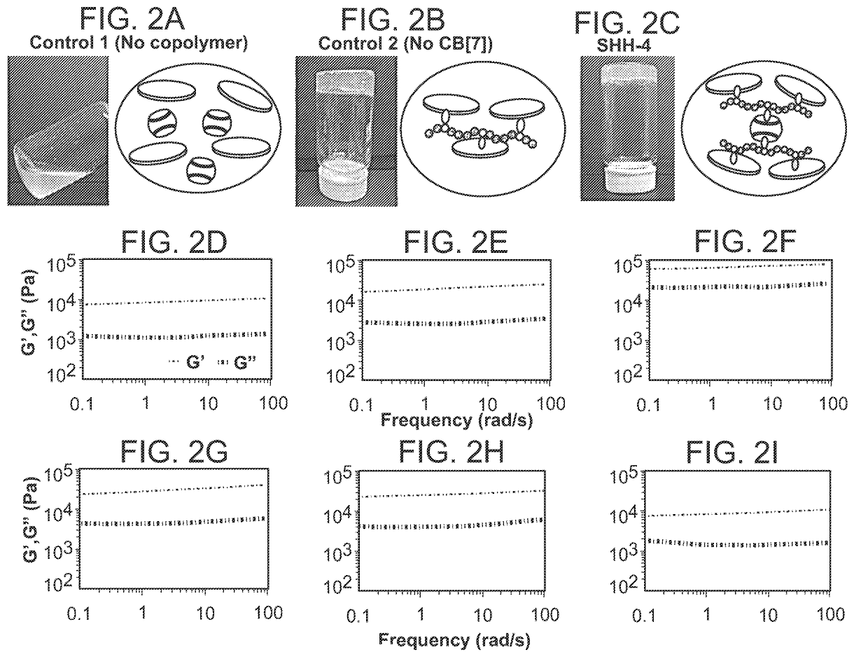

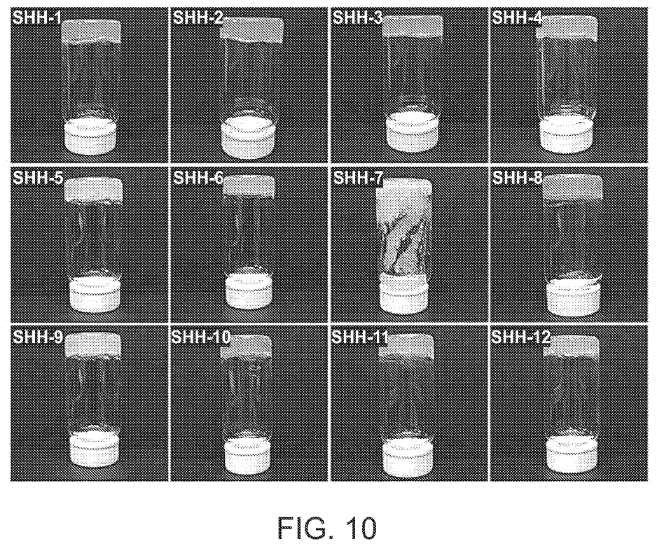

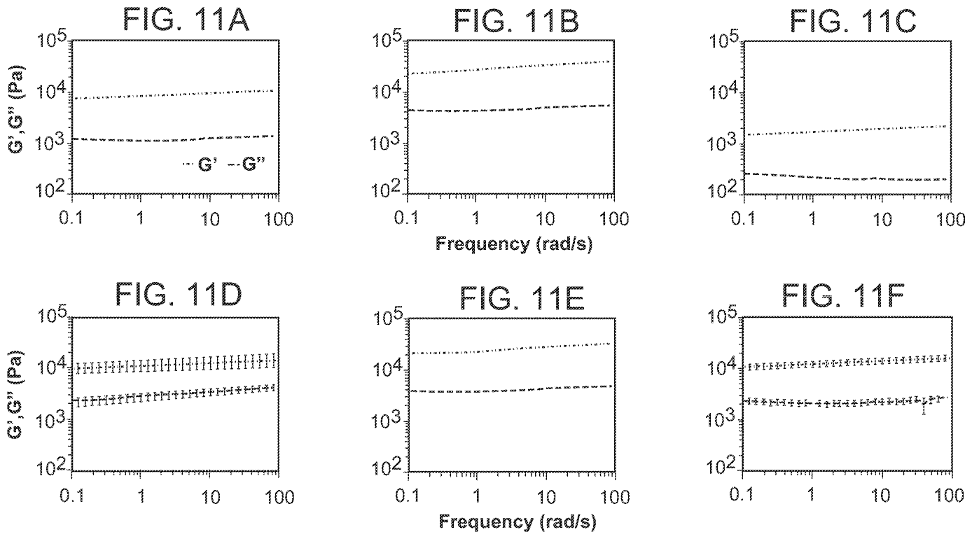

We tested a range of concentrations for each component based on previous reports with clay and dendritic molecular binders^{33,37}, supramolecular hydrogels mediated by pseudorotaxanes⁴³, and clay nanosheet hydrogels with a guanidinium-attached calix[4]arene binder⁴⁴. We chose different concentrations (wt./v %) of CNS (2, 2.5, 3, 4), CB[7] (0, 0.26, 0.52), SPA (0.06, 0.1, 0.14) and CP (0, 0.15, 0.25, 0.50) to evaluate hydrogelation (FIG. 2A-C, FIG. 10) and mechanical properties of SHHs (FIG. 2D-2I, Table 2, Table 3, FIG. 11 & 12).

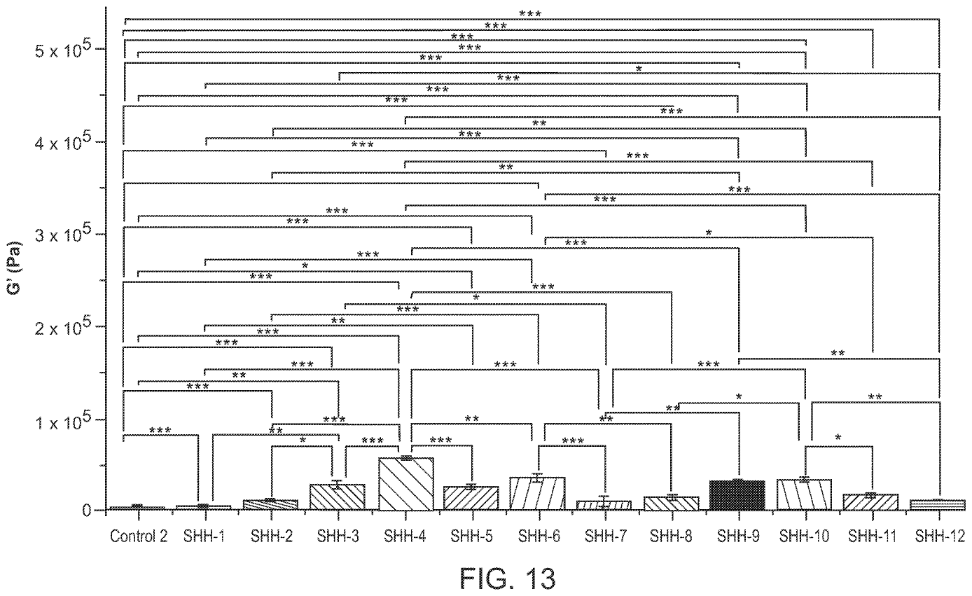

TABLE 2
Compositions of supramolecular hybrid hydrogels (SHHs) 3 separate hydrogels were
tested (N = 3) for each experiment. The data are shown as mean ± SE (N = 3).
Please see thes tatistical analyses of these measurements in FIG. 13.

| | | CB[7] | SPA | CP | | |
|---|---|---|---|---|---|---|
| | CNS | (wt./v) | (wt./v) | (wt./v) | G′ (1) | G″ (2) |
| | (wt./v) | % | % | % | (Pa) | (Pa) |
| Control-1 | 2 | 0.26 | 0.14 | 0 | N/A | N/A |
| Control-2 | 2 | 0 | 0.14 | 0.25 | 3.4 × 10³ ± 556.7 | 0.6 × 10³ ± 132.6 |
| SHH-1 | 2 | 0.26 | 0.06 | 0.15 | 3.5 × 10³ ± 791.4 | 0.5 × 10³ ± 117.5 |
| SHH-2 | 2 | 0.26 | 0.10 | 0.15 | 8.7 × 10³ ± 571.7 | 1.2 × 10³ ± 74.9 |
| SHH-3 | 2 | 0.26 | 0.14 | 0.15 | 19.4 × 10⁴ ± 1297.7 | 2.7 × 10³ ± 227.7 |
| SHH-4 | 2 | 0.26 | 0.14 | 0.25 | 5.0 × 10⁴ ± 6756.6 | 7.7 × 10³ ± 657.6 |
| SHH-5 | 2 | 0.26 | 0 | 0.25 | 1.8 × 10⁴ ± 2135.5 | 3.2 × 10³ ± 416.5 |
| SHH-6 | 2 | 0.26 | 0.14 | 0.50 | 2.7 × 10⁴ ± 3657.9 | 4.1 × 10³ ± 550.6 |
| SHH-7 | 2 | 0.52 | 0.14 | 0.50 | 1.7 × 10³ ± 70.9 | 0.2 × 10³ ± 4.0 |
| SHH-8 | 2.5 | 0.26 | 0.14 | 0.25 | 1.2 × 10⁴ ± 1768.3 | 1.7 × 10³ ± 237.7 |
| SHH-9 | 3 | 0.26 | 0.14 | 0.50 | 2.4 × 10⁴ ± 1056.2 | 3.8 × 10³ ± 40.5 |
| SHH-10 | 4 | 0.26 | 0.14 | 0.50 | 2.6 × 10⁴ ± 1797.2 | 4.3 × 10³ ± 341.3 |
| SHH-11 | 3 | 0.26 | 0.14 | 0.25 | 1.3 × 10⁴ ± 1460.6 | 2.1 × 10³ ± 249.5 |
| SHH-12 | 4 | 0.26 | 0.14 | 0.25 | 8.8 × 10³ ± 704.1 | 1.4 × 10³ ± 119.5 |

(1): Average G′ between angular frequency of 0.1-80 d/s.
(2): Average G″ between angular frequency of 0.1-80 rad/s.

TABLE 3
Young's Modulus of SHH-4 with and without CB[7].
The data are expressed as mean ± SE (N = 3).

| | Young's Modulus (kPa) |
|---|---|
| SHH-4 without CB[7] | 2.3 ± 0.23 |
| (Control 2) | 7.0 ± 0.68 |
| SHH-4 | |

Among all formulations, SHH-4—with a composition of CNS/CB[7]/SPA/CP: 2/0.610.14/0.25—formed a hydrogel in under 15 seconds and exhibited the highest mechanical properties with a storage modulus of ~50 kPa (Table 2, FIG. 2F). This corresponds to the lower end of skin modulus measurements⁴⁸⁻⁵⁰, and thus we expect SHH-4 to be intact in use as a wound dressing while still allowing comfortable movement for the patients.

As controls, a mixture of the CNS, SPA, and CB[7] without the Am-r-MATMAC CP (Control 1, FIG. 2A) and a mixture of CNS, SPA, and Am-r-MATMAC CP without CB[7] (Control 2, FIG. 2B) were prepared. No gelation was observed in control 1 (FIG. 2A). This result indicates that there is no interaction between clay and CB[7]which results in hydrogelation. On the other hand, Control 2 formed a hydrogel in 7 minutes likely due to the electrostatic interaction between the anionic surface of CNS with amine groups of Am-r-MATMAC CPs. Nevertheless, Control 2 has a lower mechanical strength and Young's modulus compared to the corresponding SHH-4 which contains the Am-r-MATMAC CP, which we discuss in the following. This result indicates that Am-r-MATMAC is valuable to obtain mechanically strong and elastic hydrogels.

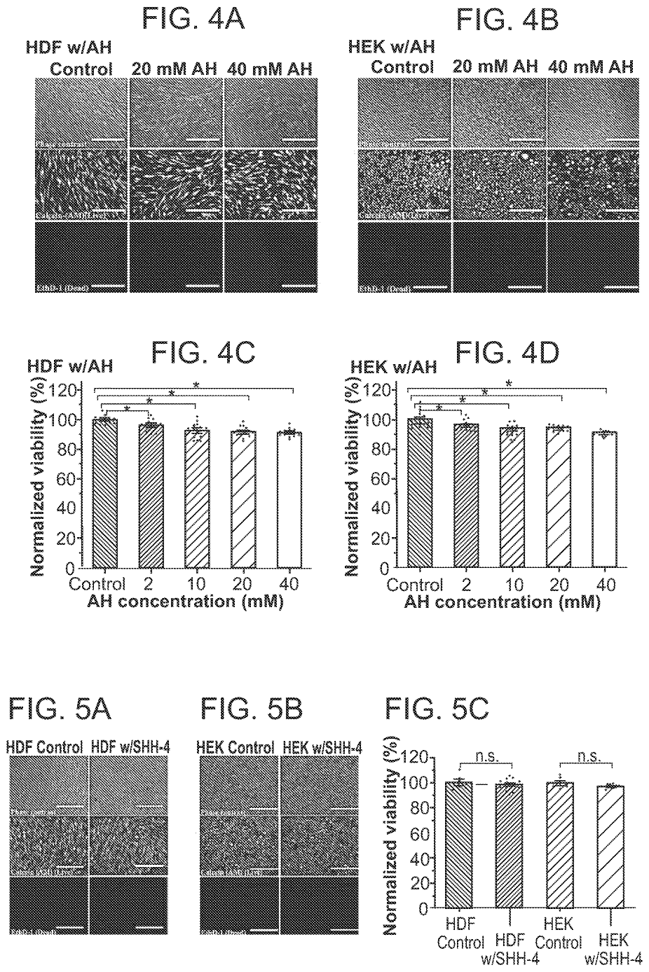

Next, we used a rheometer to evaluate the mechanical properties of various SHHs (FIG. 2D-2I, FIG. 11). The respective storage moduli (G′) and loss moduli (G″) are shown for SHHs as functions of angular frequency (∫=0.1-80 rad/s) at a fixed strain (γ) of 0.5% (FIG. 2C-2H, FIG. 11). All samples had a single plateau region in their dynamic moduli. The hydrogels demonstrated elastic response as typical hydrogels, which means G′ values were always larger than the G″ values over the entire range of frequencies. These tests showed high SPA concentrations (0.14 wt. %) are needed to obtain strong and stable SHHs. Specifically, hydrogels made with 0.14 wt. % SPA (FIG. 2F, SHH-4) has better mechanical strength than those with lower concentrations in accordance with previous report⁴⁴ (0.06 wt. % and 0.1 wt. %) (FIG. 2D, FIG. 4SA). Similarly, increasing CP concentration up to 0.25 wt./v % enhanced G′ values of SHHs (FIG. 2F). We attribute this increase in storage modulus to the interaction of the CP's cationic groups with CB[7]s and CNSs. The higher number of cationic groups led to a higher density of physical crosslinks through non-covalent and electrostatic interactions, resulting in a stronger hydrogel. Although the formulations without SPA showed a similar trend in mechanical strength with increasing CP concentrations (FIG. 2G, Table 2), the pretreatment of CNSs with SPA considerably increases the storage modulus by a factor of up to 2.7. This indicates that it is critical to pretreat CNSs with SPAs to obtain strong hydrogels^{33,43,51}. We also tested the effect of CNS concentration on mechanical strength. We observed that the mechanical properties decreased by further increasing CNS (2.5, 3, 4 wt./v %) and CP concentration (0.50 wt. %) (FIG. 2H & 2I, FIG. 11D-11E-11F). At higher CNS concentrations, we believe that the delamination of CNSs could not be effectively achieved via SPA, and as a result, electrostatic interactions between CNSs and CP decreased: thus, relatively weaker hydrogels were obtained compared to SHH-4.

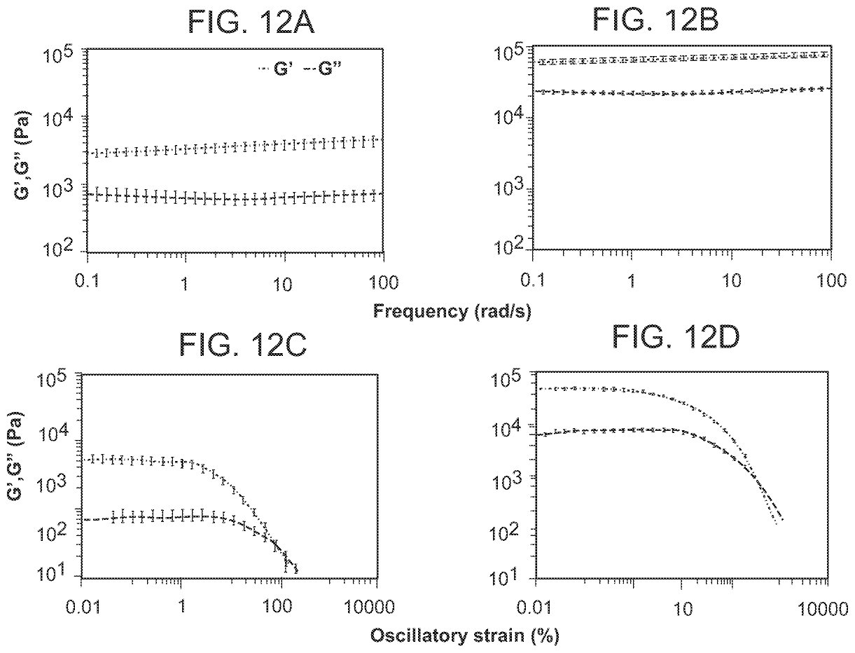

We also assessed the effects of CB[7] concentration on mechanical properties. To this end, we first compared the rheological and mechanical properties of SHH-4 and SHH-4 without CB[7], i.e., Control 2 (FIG. 12, Table 3). We found that without CB[7] (Control 2), hydrogelation took over 7 minutes as opposed to mere 15 seconds with CB[7] (SHH-4). Further, the G′ values of SHH-4 without CB[7] (Control 2) were approximately 15 times lower than SHH-4 (FIG. 12A & B). Moreover, oscillatory strain sweep measurements were performed to compare and identify the breakdown oscillatory force (FIG. 5C &D) of these hydrogels. SHH-4 without CB[7] (Control 2) collapsed from gel state to a quasi-liquid state above the critical strain (γ) of 120% (tan delta=G″/G′≈1.3) whereas SHH-4 required 4.5 times higher oscillatory force, γ=550%, (tan delta=G″/G′≈1.4) for the breakdown. Thus, the strength of SHH-4 is attributed to the supramolecular interactions between CB[7] and Am-r-MATMAC CP which is crucial to obtaining mechanically strong hydrogels. We also performed compression tests to calculate and compare Young's modulus of SHH-4 and Control 2. (Table 3). Consistent with rheological properties, we observed that Young's modulus of SHH-4 is 3 times higher than Control 2.

We observed that beyond certain concentrations, i.e., those used in SHH-4, further addition of CP and CB[7], without increasing the concentration of CNS, resulted in mechanically weaker hydrogels (SHH-6 and SHH-7). We posit that these decreases are likely due to an insufficient number of CNS that can bind to the CB[7]-CP non-covalent complexes, resulting in domains where CB[7]-CP complexes are free. Conversely, if we increase the CNS concentration beyond SHH=4, without changing the CB[7] and CP concentrations we also achieve weaker gels (SHH-8, SHH-11, SHH-12), indicating that excess CNS can also form free domains. We thus posit that the ratio between all 3 major components—CB[7], CP and CNS—might be ideal in SHH-4 which will require further assessment in future work with derivatives based on this ratio. Overall, we achieved the highest storage modulus (average G′=50 kPa>G″) with SHH-4 and selected this hydrogel for further characterization and evaluation in vitro and in vivo.

### 3.2.2. On-Demand Dissolution

The dissolution of SHHs relies on the non-covalent exchange mechanism whereby a dissolution agent (DSA) displaces the existing molecules (CPs). Adamantane (AD) or diamantane (DA) derivatives stand out as DSA candidates due to their very high guest-binding affinities with CB[7]s²⁸ ranging from 2×10¹²-7.2×10¹⁷ M⁻¹. This provides a large dynamic range for the choice of dissolution agent⁵². Here we used amantadine hydrochloride (AH)²⁸—a member of the AD family—as the dissolution agent (i.e., DSA) for dissolution. AH binds CB[7] more strongly than the cationic groups on the CP. We note that we have attempted to increase the concentration of the AH solution beyond 40 mM to reduce the dissolution time. Nevertheless, upon a preliminary screening of concentrations via in vitro viability testing, we found that AH concentrations above 40 mM can be toxic to cells in the skin niche and resulted in detachment of cells from culture surfaces (Please see section 3.3.1 for further details). Accordingly, in what follows we demonstrate dissolution tests only with 20 and 40 mM AH concentrations.

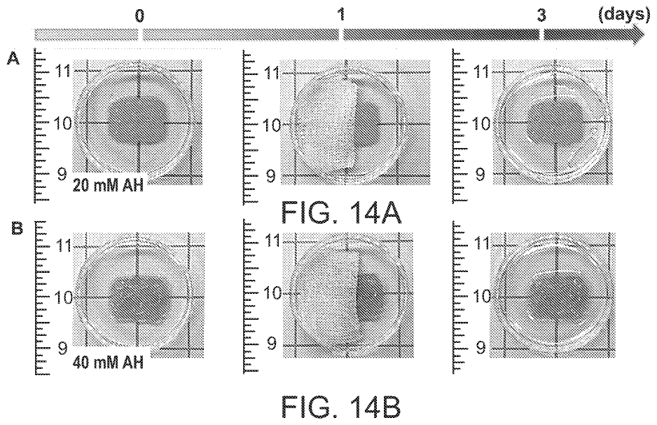

We first tested the dissolution of SHH-4 w/o CB[7] (Control 2) using 20 mM and 40 mM AH solution (FIG. 14). We did not observe any dissolution of this gel (Control 2) even after 3 days of exposure to the AH solutions. This result indicates that CB[7] is absolutely crucial for the dissolution process. Upon addition to the SHH, AH breaks the CP-CB[7]crosslinks through the non-covalent exchange mechanism. As a result, SHHs rapidly dissolved upon exposure to AH (FIG. 1B). SHH-4s were dissolved in only ~6 min with 20 mM AH (FIG. 3A) and in ~4 min with 40 mM AH (FIG. 3B). Taken together with the rheological testing results, these showed that our novel supramolecular hybrid hydrogel design can provide a mechanically tough yet quickly dissoluble dressing for easy removal and dressing changes.

### 3.2.3. Self-Healing Ability

Self-healing enables the spontaneous regeneration and repair of a hydrogel network through covalent or non-covalent interactions after mechanical damage. Therefore, the incorporation of this feature into hydrogels has gained popularity in wound treatment due to the structural stability and robustness they provide⁵³. In wound dressings, self-healing improves the durability and performance of hydrogels and provides a stable connection between the wound site and the dressing⁵⁴. This makes self-healing hydrogels a superior choice, especially in treating wounds at or near extremities (e.g., ankle, knee, and wrists).

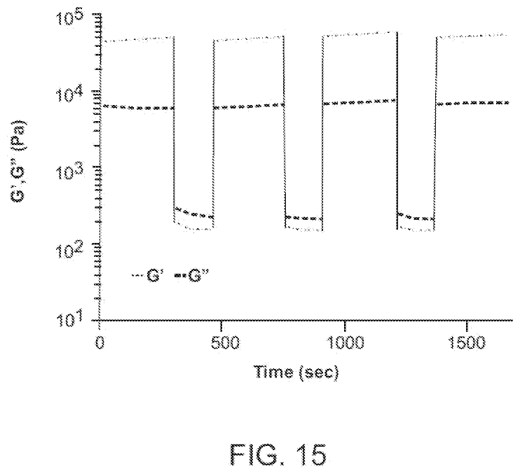

We assessed the self-healing capacity of SHH-4 by a macroscopic damage test using two pre-gelled flower-shaped SHHs stained blue (trypan blue) and red (rhodamine B) (FIG. 3C). We first cut both gels in half using a scalpel and then allowed contact between the two different colored halves to observe their self-healing ability. We found that the cut hydrogel pieces can rapidly—in about 1 min (1.2±0.1 min)—combine into flower-shaped hydrogels again without any external stimulus and the self-healed hydrogels were strong enough to be lifted (FIG. 3C). To assess the rapid recovery of SHH-4 further, we performed step-rate time-sweep measurements at a constant frequency (ω=6.0 rad/s (1.0 Hz)) (FIG. 15). SHH-4 was subjected to 0.1% strain for 300 s, then 600% strain was applied to damage the hydrogel for 150 s (quasi-liquid state, tan delta=G″/G′≈1.5). However, when the applied strain was reduced to 0.1% for recovery for another 300 s, G′ immediately recovers its initial value (tan delta=G″/G′≈0.1). The rapid self-healing ability of the SHHs likely originates from non-covalent supramolecular crosslinks between Am-r-MATMAP CP and CB[7], whereas CNSs improve their mechanical strength and stability.

Our SHHs self-heal fast compared to chemically crosslinked hydrogels⁵⁵⁻⁵⁷ which require 30 minutes to 1 hour to recover their initial value. Besides, our SHHs eliminate the need for reactive chemicals, toxic catalysts, or external stimuli for self-healing, which are used in dynamic covalent reactions (chemical crosslinking)⁵⁸. On the other hand, there are several examples of supramolecular hydrogels which can recover at rates comparable to our SHHs. However, their G′ value is 250 times⁵⁹ and 40 times¹⁹ smaller than SHH-4. Accordingly, we posit that our SHHs—with their simultaneous high mechanical strength and quick self-healing ability—constitute great candidates as wound dressings compared to the existing approaches.

### 3.2.4 pH Stability and Degree of Swelling

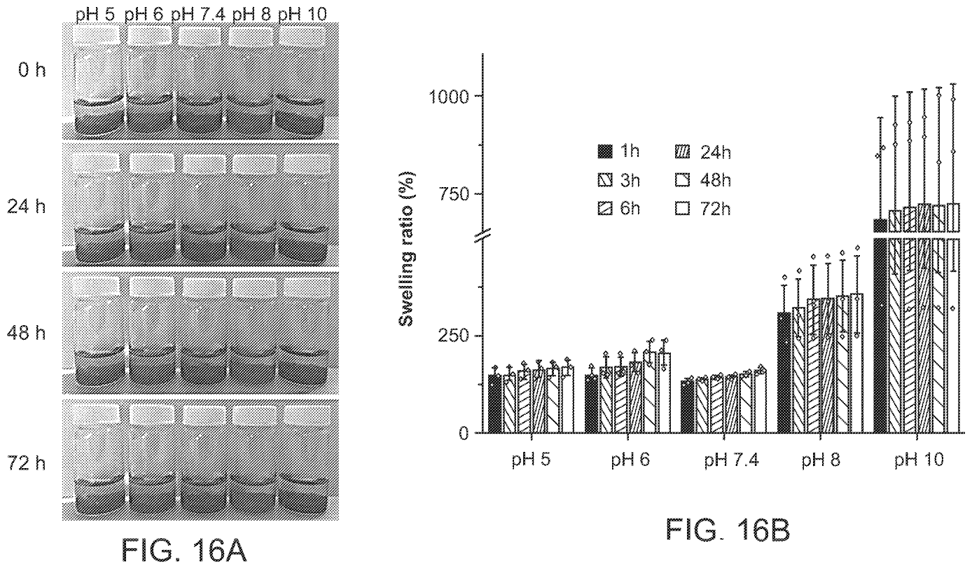

Hydrogels can absorb and hold the wound exudate. This facilitates the proliferation of fibroblasts and the migration of keratinocytes, both essential for the complete epithelialization of the wound^{47,60}. As hydrogels swell, they can trap wound debris and bacteria in the gel matrix, potentially reducing wound bioburden. Thus, here we assessed the pH stability and swelling capacity of the SHH-4 at different pH values (FIG. 16). As a swelling medium, we utilized three buffer solutions mimicking the blister fluid since its pH changes between 5-10 in second degree burns⁶¹: acidic (pH 5 acetate buffer), neutral (pH 7.4 phosphate buffer), and basic (pH 10 Tris EDTA buffer).

We first tested the effect of pH on the stability of SHHs. SHHs were maintained for 72 hours in each of these acidic, neutral, and basic solutions without any disruption. Next, we tested the swelling ratio of SHHs using different pH solutions. After exposure to acidic and neutral buffers (pH 5-7.4), SHHs swelled between 160%-200%, and swelling reached equilibrium after 72 hours (FIG. 16). In basic buffers, the degree of swelling increased up to 357.1±66.1% at pH 8 and 722.8±288.7% at pH 10 (FIG. 16) after 72 hours.

### In Vitro Biocompatibility

### In Vitro Cytotoxicity of Dissolution Agents (DSAs) and SHHs

While designing an on-demand dissoluble dressing, it is critical to evaluate the DSA toxicity. We, thus, assessed dissolution agent (DSAs) toxicity on two relevant human skin cell types, i.e., human dermal fibroblasts (HDF) and human epidermal keratinocytes (HEK). We exposed monolayer cultures of both cell types to solutions of amantadine hydrochloride (AH) and memantine hydrochloride with different concentrations (2-60 mM) for 4-10 min. We then assessed the cell viability via i) live/dead staining and ii) presto blue metabolic assay. The results are presented (FIG. 4) in comparison to control groups which were only exposed to the respective culture medium of either cell type.

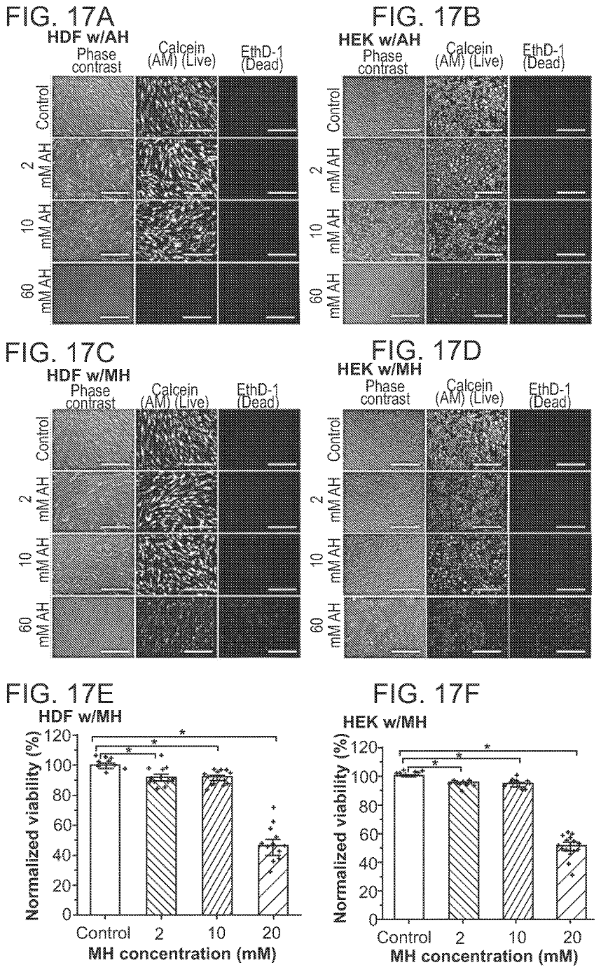

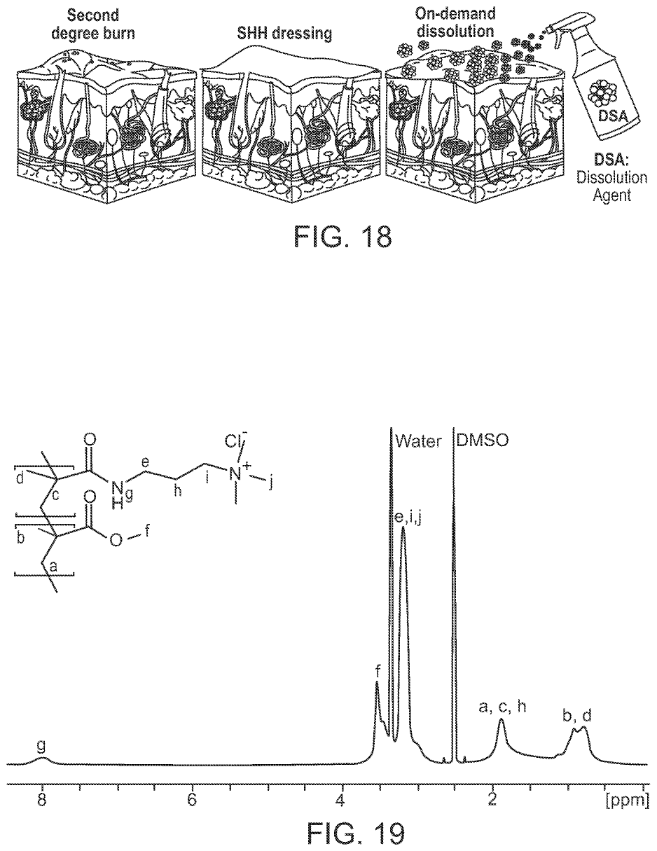

Both HDF and HEK showed high viability (>90%) in AH concentrations of 2-40 mM (FIG. 4A-4D, FIG. 16A-16B). Our results indicate that AH presents minimal adverse effects up to 40 mM on these dermal and epidermal cells. Nevertheless, a 60 mM AH concentration is toxic to the cells and results in the detachment of cells from the culture surfaces (FIG. 17A and FIG. 17B). HDF and HEK also showed high viability after exposure to 2 mM and 10 mM MH solutions, i.e., an alternative dissolution agent. However, when we increased the MH concentration to 20 mM, the viability of these cells decreased down to 44±7.6% (FIG. 17C-17F), and some of the cells detached from the surface of the wells (FIG. 17C & 17D). These results indicated that MH solution can present significant toxicity to skin cells at higher concentrations. We note that the exposure scenario we tested in these in vitro experiments is extreme. In the clinical applications, cells will likely not be directly exposed to the dissolution agent until the end stages of the dissolution, and the concentrations of such exposure will likely be reduced compared to the starting concentrations that we use here. These in vitro results posit that AH could constitute an ideal dissolution agent with minimal adverse effects in further testing and clinical use.

To ensure the use of the SHHs as potential burn dressings, we tested in vitro cytotoxicity of the SHH-4 hydrogel with both HDF and HEK. SHH disks (4 mm diameter, 2 mm thickness) were introduced into the culture and incubated for 24 hrs. We then assessed the cell viability via i) live/dead staining and ii) presto blue metabolic assay. The results are presented (FIG. 5) in comparison to control groups (culture media). The viability of HDF (98.5±4.2%) and HEK (94.3±1.4%) exposed to the SHH did not differ from controls (100±5%) after 24 hrs. (FIG. 5C). The introduction of SHHs led to no visible or significant differences in cell viability or morphology.

### In Vitro Cytokine Secretion of SHHs

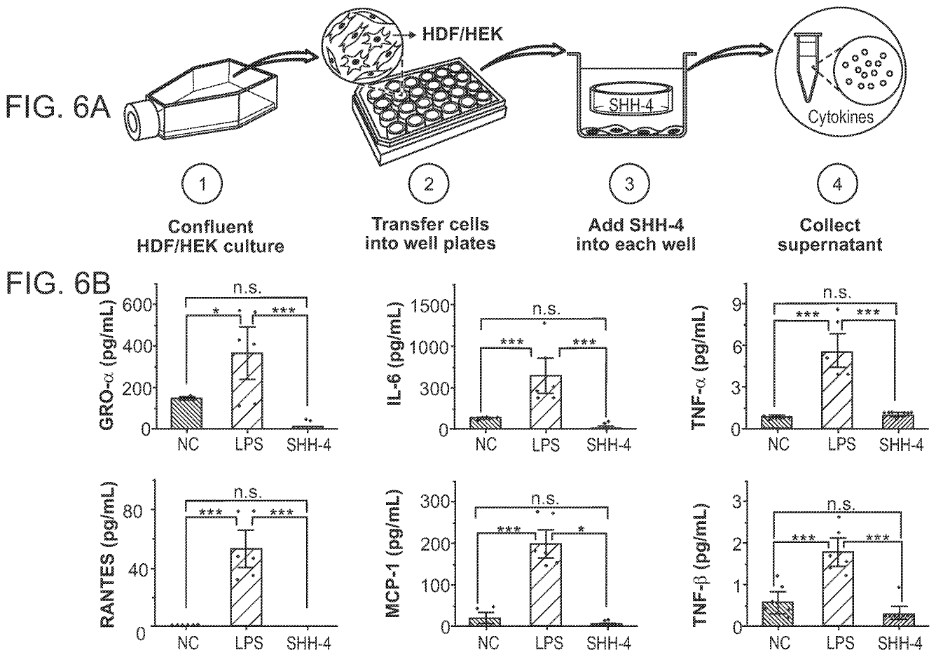

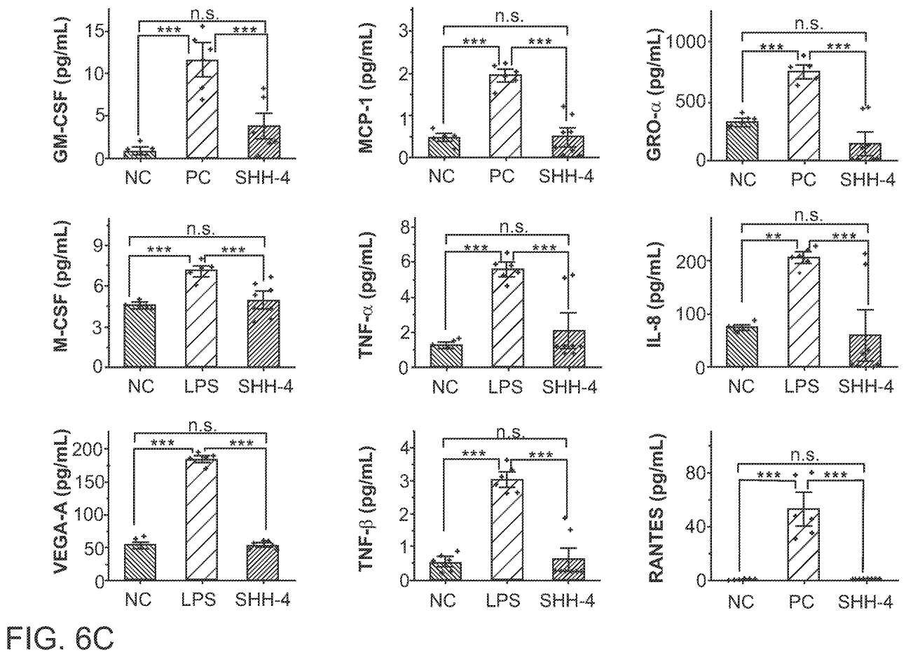

Cytokines are soluble extracellular proteins or glycoproteins that facilitate cell-cell communication, and activation of the immune and inflammatory system⁶². A design criterion for our SHHs is to not elicit any immune or inflammatory response beyond normal wound healing response. Thus, as a preliminary approach, we assessed the relative secretion levels of cytokine and growth factors of in vitro HDF and HEK cells in response to SHH-4 (FIG. 6A-6C). We profiled a panel of 48 cytokines and growth factors but only present those where the positive control (Lipopolysaccharide, LPS) elicited a substantial regulation. Importantly, we observed that the expression of various inflammatory cytokines growth-regulated oncogene)-alpha (GRO-α), Regulated on Activation Normal T Expressed and Secreted (RANTES), Interleukin-6 (IL-6), Monocyte chemoattractant protein-1 (MCP-1), Tumour Necrosis Factor alpha (TNF-α), Tumour Necrosis Factor beta (TNF-β) for HDF, and Granulocyte-macrophage colony-stimulating factor (GM-CSF), Macrophage colony-stimulating factor (M-CSF), Vascular endothelial growth factor A (VEGF-A), Monocyte chemoattractant protein-1 (MCP-1), TNF-α, TNF-β, GRO-α, Interleukin-8 (IL-8), RANTES for HEK did not differ from media only controls and was significantly lower than the positive controls exposed to lipopolysaccharide (LPS) (FIG. 6B & C). This lack of cytokine secretion regulation in the presence of SHHs demonstrated that HDF and HEK are not activated by the SHHs in our in vitro experiments.

### In Vivo Biocompatibility

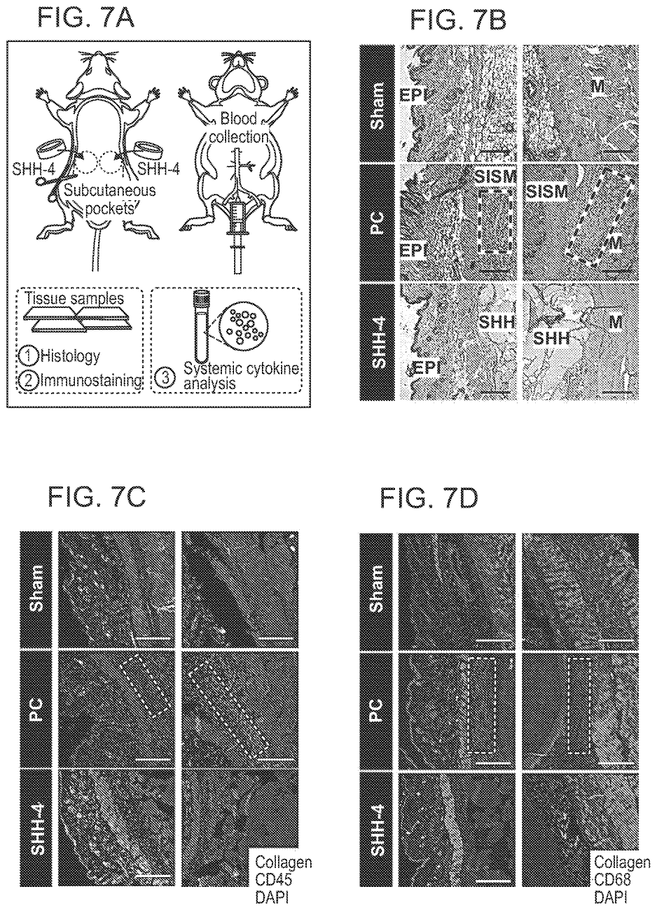

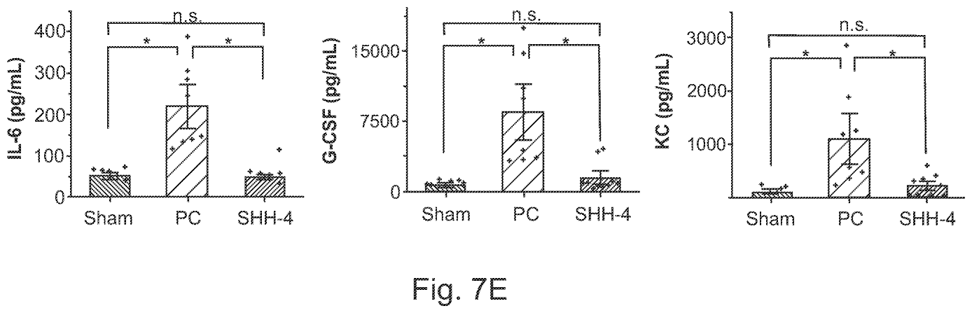

The biocompatibility of the hydrogel with the skin tissue or other tissues it is intended for is essential for efficient and timely wound healing. A biocompatible hydrogel should integrate itself on the tissue with limited inflammatory response beyond normal wound healing, and preferably without “foreign body reactions” such as accumulating giant cells, macrophages, and leukocytes. Moreover, it should provide healing with minimal to no fibrosis⁶³⁻⁶⁵. To assess the in vivo biocompatibility of our best-performing SHH, we subcutaneously implanted the SHH-4 hybrid hydrogels (8 mm diameter, 2 mm thickness) into the dorsal subcutaneous pockets of mice. Negative control (NC; sham, no implantation) and positive control (PC); small intestinal submucosa implantation to the dorsal pocket) experiments were also conducted simultaneously. 48 hours after implantation, the surrounding tissue of the implantation site was collected for histological and immunostaining analyses and blood was drawn from the inferior vena cava for systemic cytokine analysis (FIG. 7A).

### Histological Analysis

Histological analyses were performed to evaluate the morphology and immune cell infiltration under the subcutaneous tissue. In the PC group, immune cells infiltrated the interstitial area and muscular tissues around the implants as indicated with a dashed rectangle (FIG. 7B). In stark contrast with this drastic infiltration in the PC group, the SHH4 group showed limited infiltration at levels similar to the sham (NC) group. These results indicate that SHH-4 implantation did not invoke any infiltration of immune cells under the subcutaneous tissue compared to the sham control (FIG. 7B).

### Immunofluorescence Staining

Immunohistochemistry was performed to assess the infiltration of different immune and inflammatory cells in the subcutaneous tissue and hydrogel, including general leukocytes (CD45), and macrophages (CD68) (FIG. 7C & 7D). CD45-positive leukocytes (including neutrophils) are immune cells which infiltrate and participate in the immune rejection and implantation failures of skin xenografts⁶⁶ and allografts⁶⁷. CD68-positive macrophages have also been reported in the rejection of skin grafts⁶⁸, and they are often considered as therapeutic target to promote the survival of transplanted organs due to their destructive effect on the graft tissue⁶⁹. In the sham group, no CD45 and CD68 positive immune cells were observed in the surrounding tissue of the subcutaneous pocket (FIG. 7C & 7D). We note that the anti-CD68 antibodies also label muscular tissue, but this labeling was excluded from the analysis based on the distinct morphology of the macrophages. High numbers of CD45 and CD68 positive cells were identified in the interstitial area around the implants in the PC group. In contrast, only a few CD45 and CD68 positive immune cells were observed around the SHH-4 implants which indicated limited leukocyte and macrophage infiltration. These results are in good agreement with the H&E stained sections. We, thus, conclude that the SHH-4 implants do not trigger any significant immune responses histologically (levels are similar to the sham group and significantly lower than the PC group).

### Systemic Cytokine Analysis

In addition to histological and immunostaining analyses, we quantified the cytokine levels in the circulating plasma to evaluate whether the implantation of SHH-4 results in any alterations for systemic cytokine levels (FIG. 7E). We profiled a panel of 48 cytokines and growth factors but only present those where the PC elicited a substantial regulation. Among the 48 cytokines profiled, the PC group mice showed elevated levels in interleukin 6 (IL-6), granulocyte colony-stimulating factor (G-CSF), and keratinocyte chemoattractant (KC) compared to sham control and SHH-4 implants. The elevated expression of cytokines IL-6, G-CSF, and KC in the PC group are associated with immune rejection by the skin tissue. IL-6 is synthesized at the local lesion to initiate inflammation⁵² and is elevated to modulate inflammatory rejection and dermal fibroblasts activity in response to xenobiotic materials, such as breast silicone implants both in vitro and in vivo⁷⁰. G-CSF is known for its regulatory role in promoting survival, proliferation, differentiation, and function of neutrophils, while affecting T cell and dendritic cell functions^{71,72}. The elevation of G-CSF level correlated with highly mobilized neutrophil activities around the implant of the PC group (FIG. 7B). KC (or CXCL-1 in humans), is a mediator for immune response and a potent attractant for neutrophils and other non-hematopoietic cells to the injury or infection site⁷³, the overexpression of KC has been found to promote higher and prolonged immune cell influx in the foreign body reaction of implanted materials in the skin tissue⁷⁴. The low levels of these cytokines in the SHH-4 group indicated a limited activation of the neutrophils and fibroblasts that are comparable to the sham group and significantly lower than the positive group. This indicates that SHH-4 does not elicit any considerable immune responses beyond what is necessary for normal wound healing response (i.e., the response of the sham group). As with the local H&E and immunostaining analyses, the systemic expression of these cytokines was significantly lower in the SHH-4 implanted mice compared to the PC group, and comparable to the expression in that of the sham group. We thus conclude that the SHH-4 did not elicit a significant systemic cytokine response in mice upon implantation.

### Example 2

### Synthesis of Poly (methyl methacrylate-random-3-(methacryloylamino)propyl] trimethylammonium chloride) (P-MMA-r-MATMAC)—Abbreviated as PMM

The random copolymer poly(methyl methacrylate-random-(3-methacryloylamino) propyl trimethylammonium chloride) (PMM) was synthesized following a free radical polymerization. Briefly, 5.5 mL MATMAC and 2.25 g MMA were dissolved in 20 mL of dimethyl sulfoxide (DMSO) in a 100 mL round bottom flask at room temperature. 0.005 g of the initiator AIBN was added after dissolution. The reaction mixture was purged with nitrogen for 30 min to purge any dissolved oxygen. The flask was then placed in an oil bath set to 60 °C, while stirring at 300 rpm for 20 hours. The flask was removed from the oil bath and unsealed, and 0.5 g of MEHQ was added to terminate the reaction. The reaction mixture was then precipitated in acetone and purified by stirring three fresh portions of 5% distilled water and 95% acetone by volume for at least 3 hours to eliminate any remaining unreacted monomer. The attained solid polymer was dried for two days under a fume hood and two more days in a vacuum oven at 50 °C. The product yield was 35% and calculated from the ratio of the mass of the product copolymer to the mass of the monomers used. The chemical composition of the copolymer was obtained by 1H-NMR (Bruker Avance III 500 MHz spectrometer, DMSO-d6) (FIG. 19). The calculated composition was: 58 wt % MATMAC, and 42 wt % MMA.

### Synthesis of Poly (2-methacryloyloxyethyl phosphorylcholine)—Abbreviated as PMPC

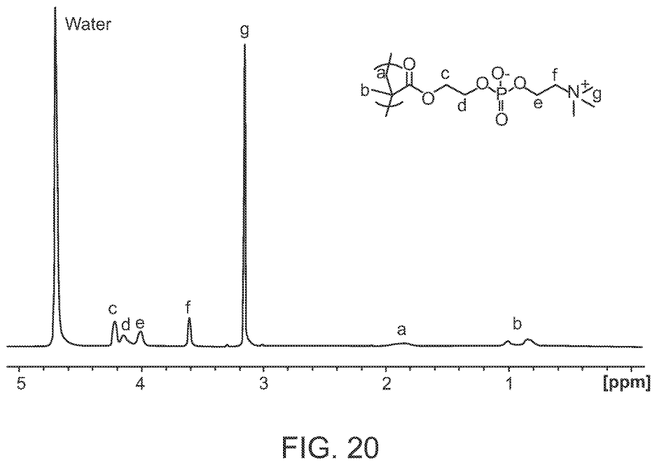

The polymer poly(2-methacryloyloxyethyl phosphorylcholine) (PMPC) was synthesized following a free radical polymerization. Briefly, 5 g MPC was dissolved in 40 mL of dime-thyl sulfoxide in a 100 mL round bottom flask at room temperature. 0.005 g of the initia-tor AIBN was added after dissolution. The reaction mixture was purged with nitrogen for 30 min to purge any dissolved oxygen. The flask was then placed in an oil bath set to 60 °C, while stirring at 300 rpm for 20 hours. The flask was removed from the oil bath and unsealed, and 0.5 g of MEHQ was added to terminate the reaction. The reaction mixture was then precipitated in acetone and purified by stirring three fresh portions of 8% methanol and 92% acetone by volume for at least 3 hours to eliminate any remaining unreacted monomer. The attained solid polymer was dried for two days under a fume hood and two more days in a vacuum oven at 50 °C. The product yield was 45% and calculated from the ratio of the mass of the product polymer to the mass of the monomer used. The chemical composition of the polymer was verified by 1H-NMR (Bruker Avance III 500 MHz spectrometer, using D2O) (FIG. 20).

Fabricating Mechanically Weak Control Hydrogels without Polymers Described as Component 1, and Mechanical Characterizations of these Hydrogels Using a Rheometer

Hydrogel fabrication description for Control-1: first a 5 wt % solution of CNS in water, 0.28 wt % solution of SPA in water, and a 2 wt % solution of CB[7] in water were pre-pared. This was followed by the preparation of a 2 wt % CNS, 0.26 wt % CB[7], and 0.14 wt % SPA mixture (Table 1) by adding 1 mL of CNS solution, 0.5 mL of SPA solution, 0.2 mL of CB[7] solution, and 0.3 mL of DIW to achieve a final sample amount of 2 mL. The solution was vortexed for approximately 30 s to achieve complete dissolution. With-in 5 min, the mixture formed a weak hydrogel.

Hydrogel fabrication description for Control-3,4,5: first a 5 wt % solution of CNS in water was prepared. This was followed by the preparations of a 2-3.85 wt % CNS mixture (Table 1) by adding 1-1.54 mL of CNS solution and 0.46-1 mL of DIW to achieve final sample amounts of 2 mL. The solution was vortexed for approximately 30 s to achieve complete dissolution. Within 5 min, the mixture formed a weak hydrogel.

The rheological properties of control hydrogels were measured at 25 °C using a rheometer (TA Instruments. Discovery HR 20 Rheometer, New Castle, DE) fitted with parallel plates (diameter 20 mm). The gap at the apex of the para-plate was set to 1 mm. The samples (=15 mm dia., Z 3 mm thickness) were placed between the para-plate and the platform with special care to avoid water evaporation. Dynamic frequency sweep tests (0.1-80 rad/s) at a fixed strain of 0.5% were measured to determine the storage modulus (G′) and loss modulus (G″) of hydrogels (Table 1). At least two samples of each hydrogel were tested for rheological tests.

Compositions and rheological test results averages for control hydrogels.

| | CNS | CB[7] | SPA | PAM | PMPC | | |
|---|---|---|---|---|---|---|---|
| Sample | (wt./v) | (wt./v) | (wt./v) | (wt./v) | (wt./v) | G′ (1) | G″ (2) |
| Units | % | % | % | % | % | (Pa) | (Pa) |
| Control-1 | 2 | 0.26 | 0.14 | 0 | 0 | N/A | N/A |
| Control-3 | 2.5 | 0 | 0 | 0 | 0 | 12.35 ± 4.90 | 5.14 ± 2.45 |
| Control-4 | 3.2 | 0 | 0 | 0 | 0 | 290.1 ± 35.1 | 22.7 ± 1.78 |
| Control-5 | 3.85 | 0 | 0 | 0 | 0 | 698.5 ± 13.4 | 39.47 ± 0.04 |

Fabricating SHH Hydrogels Using PAM and Mechanical Characterizations of these Hydrogels Using a Rheometer

Hydrogel fabrication description for Control-2: first a 5 wt % solution of CNS in water. 0.28 wt % solution of SPA in water, and a 2.4 wt % solution of copolymer PAM in water were prepared. This was followed by the preparation of a 2 wt % CNS, 0.14 wt % SPA, and 0.25 wt % PAM mixture (Table 2) by adding 1 mL of CNS solution and 1 mL of SPA solution. After using the vortex for 30 s, 0.21 mL of PAM solution was added to achieve a final sample amount of 2.2 mL. The solution was vortexed for another 30 s to achieve complete dissolution. Within 5 min, the mixture formed a hydrogel.

Hydrogel fabrication description for Control-6 to Control-11: first a 5 wt % solution of CNS in water and a 2.5 wt % solution of copolymer PAM in water were prepared. This was followed by the preparation of a 2.5-3.85 wt % CNS and 0.25-0.5 wt % PAM mixture (Table 2) by adding 1-1.54 mL of CNS solution and 0.26-0.8 mL of DIW. After using the vortex for 30 s, 0.2-0.4 mL of PAM solution was added to achieve a final sample amount of 2 mL. The solution was vortexed for another 30 s to achieve complete dissolution. Within 5 min, the mixture formed a hydrogel.

Hydrogel fabrication description for SHH-1 to SHH-12: first a 4-5 wt % solution of CNS in water, 2.6 wt % solution of CB[7], 0.28-0.56 wt % solution of SPA in water, and a 2.4-4 wt % solution of copolymer PAM in water were prepared. This was followed by the preparation of a 2-4 wt % CNS, 0.26-0.52 wt % CB[7], 0.06-0.14 wt % SPA, and 0.25-0.5 wt % PAM mixture (Table 2) by adding 1-2 mL of CNS solution, 0.6-1.2 mL of SPA solution, and 0.2 mL of CB[7] solution. After using the vortex for 30 s and waiting 10 min, 0.2-0.5 mL of PAM solution was added to achieve a final sample amount of 2 mL. The solution was vortexed for another 30 s to achieve complete dissolution. Within 5 min, the mixture formed a hydrogel.

Hydrogel fabrication description for SHH-13 to SHH-21: first a 5-5.8 wt % solution of CNS in water, 2 wt % solution of CB[7], and a 2.5-4 wt % solution of copolymer PAM in water were prepared. This was followed by the preparation of a 2.5-3.85 wt % CNS, 0.087-0.5 wt % CB[7], and 0.25-0.5 wt % PAM mixture (Table 2) by adding 1-1.54 mL of CNS solution, 0-0.43 mL of DIW, and 0.087-0.5 mL of CB[7] solution. After using the vortex for 30 s and waiting 10 min, 0.2-0.5 mL of PAM solution was added to achieve a final sample amount of 2 mL. The solution was vortexed for another 30 s to achieve complete dissolution. Within 5 min, the mixture formed a hydrogel.

The rheological properties of control hydrogels and PAM containing SHH hydrogels were measured at 25 °C using a rheometer (TA Instruments, Discovery HR 20 Rheometer, New Castle, DE) fitted with parallel plates (diameter 20 mm). The gap at the apex of the para-plate was set to 1 mm. The samples (≈15 mm dia.≈3 mm thickness) were placed between the para-plate and the platform with special care to avoid water evaporation. Dynamic frequency sweep tests (0.1-80 rad/s) at a fixed strain of 0.5% were measured to determine the storage modulus (G′) and loss modulus (G″) of hydrogels (Table 2). At least two samples of each hydrogel were tested for rheological tests.

TABLE 2
Compositions and rheological test results averages for control and PAM containing SHH hydrogels.

| | CNS | CB[7] | SPA | PAM | | |
|---|---|---|---|---|---|---|
| Sample | (wt./v) | (wt./v) | (wt./v) | (wt./v) | G′ (1) | G″ (2) |
| Units | % | % | % | % | (Pa) | (Pa) |
| Control-2 | 2 | 0 | 0.14 | 0.25 | 3.4 × 10³ ± | 0.6 × 10³ ± |
| | | | | | 556.7 | 132.6 |
| Control-6 | 2.5 | 0 | 0 | 0.25 | 2524.21 | 336.99 |
| Control-7 | 3.2 | 0 | 0 | 0.25 | 3720.1 ± | 725.785 ± |
| | | | | | 760.1 | 195.9 |
| Control-8 | 3.85 | 0 | 0 | 0.25 | 4189.5 ± | 550.835 ± |
| | | | | | 221.2 | 17.24 |
| Control-9 | 2.5 | 0 | 0 | 0.5 | 4404.4 ± | 932.995 ± |
| | | | | | 576.5 | 120.4 |
| Control-10 | 3.2 | 0 | 0 | 0.5 | 5471.7 ± | 1015.265 ± |
| | | | | | 430.3 | 34.24 |
| Control-11 | 3.85 | 0 | 0 | 0.5 | 7230.4 ± | 982.89 ± |
| | | | | | 506.0 | 118.5 |
| SHH-1 | 2 | 0.26 | 0.06 | 0.15 | 3.5 × 10³ ± | 0.5 × 10³ ± |
| | | | | | 791.4 | 117.5 |
| SHH-2 | 2 | 0.26 | 0.1 | 0.15 | 8.7 × 10³ ± | 1.2 × 10³ ± |
| | | | | | 571.7 | 74.9 |
| SHH-3 | 2 | 0.26 | 0.14 | 0.15 | 19,4 × 10⁴ ± | 2.7 × 10³ ± |
| | | | | | 1297.7 | 227.7 |
| SHH-4 | 2 | 0.26 | 0.14 | 0.25 | 5.0 × 10⁴ ± | 7.7 × 10³ ± |
| | | | | | 6756.6 | 657.6 |
| SHH-5 | 2 | 0.26 | 0 | 0.25 | 1.8 × 10⁴ ± | 3.2 × 10³ ± |
| | | | | | 2135.5 | 416.5 |
| SHH-6 | 2 | 0.26 | 0.14 | 0.5 | 2.7 × 10⁴ ± | 4.1 × 10³ ± |
| | | | | | 3657.9 | 550.6 |
| SHH-7 | 2 | 0.52 | 0.14 | 0.5 | 1.7 × 10³ ± | 0.2 × 10³ ± |
| | | | | | 70.9 | 4.0 |
| SHH-8 | 2.5 | 0.26 | 0.14 | 0.25 | 1.2 × 10⁴ ± | 1.7 × 10³ ± |
| | | | | | 1768.3 | 237.7 |
| SHH-9 | 3 | 0.26 | 0.14 | 0.5 | 2.4 × 10⁴ ± | 3.8 × 10³ ± |
| | | | | | 1056.2 | 40.5 |
| SHH-10 | 4 | 0.26 | 0.14 | 0.5 | 2.6 × 10⁴ ± | 4.3 × 10³ ± |
| | | | | | 1797.2 | 341.3 |
| SHH-11 | 3 | 0.26 | 0.14 | 0.25 | 1.3 × 10⁴ ± | 2.1 × 10³ ± |
| | | | | | 1460.6 | 249.5 |
| SHH-12 | 4 | 0.26 | 0.14 | 0.25 | 8.8 × 10³ ± | 1.4 × 10³ ± |
| | | | | | 704.1 | 119.5 |
| SHH-13 | 3.2 | 0.26 | 0 | 0.25 | 37005.2 ± | 5964.1 ± |
| | | | | | 4017.38 | 407.62 |
| SHH-14 | 3.85 | 0.26 | 0 | 0.25 | 27018.7 ± | 4757.45 ± |
| | | | | | 4299.95 | 739.59 |
| SHH-15 | 2.5 | 0.26 | 0 | 0.25 | 40330.7 ± | 6701.166 ± |
| | | | | | 19845.0 | 2881.6 |
| SHH-16 | 2.5 | 0.26 | 0 | 0.5 | 38669.3 ± | 6715.435 ± |
| | | | | | 17928.7 | 3079.5 |
| SHH-17 | 3.2 | 0.26 | 0 | 0.5 | 51446.4 ± | 7425.805 ± |
| | | | | | 17240.2 | 2577.8 |
| SHH-18 | 3.85 | 0.26 | 0 | 0.5 | 27671.3 ± | 3907.52 ± |
| | | | | | 118.199 | 27.322 |
| SHH-19 | 3.2 | 0.5 | 0 | 0.5 | 8247.18 ± | 1175.81 ± |
| | | | | | 110.838 | 194.01 |

Fabricating SHH Hydrogels Using PMM and Mechanical Characterizations of these Hydrogels Using a Rheometer

Hydrogel fabrication description for SHH-34: first a 5 wt % solution of CNS in water, 2 wt % solution of CB[7], and a 2.5 wt % solution of copolymer PEI in water were prepared. This was followed by the preparation of a 3.2 wt % CNS, 0.26 wt % CB[7], and 0.25 wt % PMM mixture (Table 3) by adding 1.28 mL of CNS solution, 0.26 mL of DIW, and 0.26 mL of CB[7] solution. After using the vortex for 30 s and waiting 10 min, 0.2 mL of PMM solution was added to achieve a final sample amount of 2 mL. The solution was vortexed for another 30 s to achieve complete dissolution. Within 5 min, the mixture formed a hydrogel.

The rheological properties of PMM containing SHH hydrogels were measured at 25 °C using a rheometer (TA Instruments, Discovery HR 20 Rheometer, New Castle, DE) fitted with parallel plates (diameter 20 mm). The gap at the apex of the para-plate was set to 1 mm. The samples (z 15 mm dia., z 3 mm thickness) were placed between the para-plate and the platform with special care to avoid water evaporation. Dynamic frequency sweep tests (0.1-80 rad/s) at a fixed strain of 0.5% were measured to determine the storage modulus (G′) and loss modulus (G″) of hydrogels (Table 3). At least two samples of each hydrogel were tested for rheological tests.

Compositions and rheological test results averages for PMM containing SHH hydrogels.

| | CNS | CB[7] | SPA | PMM | | |
|---|---|---|---|---|---|---|
| Sample | (wt./v) | (wt./v) | (wt./v) | (wt./v) | G′ (1) | G″ (2) |
| Units | % | % | % | % | (Pa) | (Pa) |
| SHH-34 | 3.2 | 0.26 | 0 | 0.25 | 23517.36 ± | 2866.314 ± |
| | | | | | 1147.56 | 214.427 |

Fabricating SHH Hydrogels Using PMPC and Mechanical Characterizations of these Hydrogels Using a Rheometer

Hydrogel fabrication description for Control-12: first a 4 wt % solution of CNS in water, 0.28 wt % solution of SPA in water, and a 2.4 wt % solution of PMPC in water were prepared. This was followed by the preparation of a 2 wt % CNS, 0.14 wt % SPA, and 0.25 wt % PMPC mixture (Table 4) by adding 1 mL of CNS solution and 1 mL of SPA solution. After using the vortex for 30 s, 0.2 mL of PMPC solution was added to achieve a final sample amount of 2.2 mL. The solution was vortexed for another 30 s to achieve complete dissolution. Within 5 min, the mixture formed a hydrogel.

Hydrogel fabrication description for Control-13 to Control-20: first a 4-5 wt % solution of CNS in water and a 2.5 wt % solution of copolymer PMPC in water were prepared. This was followed by the preparation of a 2-3.85 wt % CNS and 0.25-0.5 wt % PAM mixture (Table 4) by adding 1-1.54 mL of CNS solution and 0.26-0.8 mL of DIW. After using the vortex for 30 s, 0.2-0.4 mL of PMPC solution was added to achieve a final sample amount of 2 mL. The solution was vortexed for another 30 s to achieve complete dissolution. Within 5 min, the mixture formed a hydrogel.

Hydrogel fabrication description for SHH-22: first a 4 wt % solution of CNS in water, 2.6 wt % solution of CB[7], 0.56 wt % solution of SPA in water, and a 2.5 wt % solution of PMPC in water were prepared. This was followed by the preparation of a 2 wt % CNS, 0.26 wt % CB[7], 0.14 wt % SPA, and 0.25 wt % PMPC mixture (Table 4) by adding 1 mL of CNS solution, 0.1 mL of DIW, 0.5 mL of SPA solution, and 0.2 mL of CB[7] solution. After using the vortex for 30 s and waiting 10 min. 0.2 mL of PMPC solution was added to achieve a final sample amount of 2 mL. The solution was vortexed for another 30 s to achieve complete dissolution. Within 5 min, the mixture formed a hydrogel.

Hydrogel fabrication description for SHH-23 to SHH-32: first a 5-5.8 wt % solution of CNS in water, 2 wt % solution of CB[7], and a 2.5-4 wt % solution of PMPC in water were prepared. This was followed by the preparation of a 2.5-4.47 wt % CNS, 0.087-0.5 wt % CB[7], and 0.25-0.5 wt % PMPC mixture (Table 4) by adding 1-1.54 mL of CNS solution, 0-0.43 mL of DIW, and 0.087-0.5 mL of CB[7] solution. After using the vortex for 30 s and waiting 10 min, 0.2-0.5 mL of PMPC solution was added to achieve a final sample amount of 2 mL. The solution was vortexed for another 30 s to achieve complete dissolution. Within 5 min, the mixture formed a hydrogel.

The rheological properties of control hydrogels and PAM containing SHH hydrogels were measured at 25 °C using a rheometer (TA Instruments, Discovery HR 20 Rheometer, New Castle, DE) fitted with parallel plates (diameter 20 mm). The gap at the apex of the para-plate was set to 1 mm. The samples (≈15 mm dia.≈3 mm thickness) were placed between the para-plate and the platform with special care to avoid water evaporation. Dynamic frequency sweep tests (0.1-80 rad/s) at a fixed strain of 0.5% were measured to determine the storage modulus (G′) and loss modulus (G″) of hydrogels (Table 4). At least two samples of each hydrogel were tested for rheological tests.

TABLE 4
Compositions and rheological test results averages for control
and PMPC containing SHH hydrogels.

| | CNS | CB[7] | SPA | PMPC | | |
|---|---|---|---|---|---|---|
| Sample | (wt./v) | (wt./v) | (wt./v) | (wt./v) | G′ (1) | G″ (2) |
| Units | % | % | % | % | (Pa) | (Pa) |
| Control- | 2 | 0 | 0.14 | 0.25 | 839.2 ± | 90.40 ± |
| 12 | | | | | 34.36 | 10.18 |
| Control- | 2.5 | 0 | 0 | 0.25 | 116.3 ± | 17.3 ± |
| 13 | | | | | 101.7 | 9.30 |
| Control- | 3.2 | 0 | 0 | 0.25 | 268.0 ± | 23.66 ± |
| 14 | | | | | 42.2 | 3.18 |
| Control- | 3.85 | 0 | 0 | 0.25 | 1085.9 ± | 71.54 ± |
| 15 | | | | | 38.0 | 2.84 |
| Control- | 2.5 | 0 | 0 | 0.5 | 109.9 ± | 20.275 ± |
| 16 | | | | | 40.1 | 2.32 |
| Control- | 3.2 | 0 | 0 | 0.5 | 489.0 ± | 43.18 ± |
| 17 | | | | | 52.2 | 3.74 |
| Control- | 3.85 | 0 | 0 | 0.5 | 1108.0 ± | 86.605 ± |
| 18 | | | | | 20.0 | 0.06 |
| Control- | 2 | 0 | 0 | 0.25 | 69.67 ± | 11.32 ± |
| 19 | | | | | 7.00 | 0.261 |
| Control- | 3.6 | 0 | 0 | 0.25 | 1467.27 ± | 103.64 ± |
| 20 | | | | | 111.52 | 9.545 |
| SHH-20 | 2 | 0.26 | 0.14 | 0.25 | 2792.37 ± | 253.32 ± |
| | | | | | 698.9 | 57.53 |
| SHH-21 | 3.2 | 0.26 | 0 | 0.25 | 1891.66 ± | 276.35 ± |
| | | | | | 68.0943 | 10.210 |
| SHH-22 | 3.85 | 0.26 | 0 | 0.25 | 2784.67 ± | 403.26 ± |
| | | | | | 33.0501 | 16.136 |
| SHH-23 | 2.5 | 0.26 | 0 | 0.25 | 1773.53 ± | 233.455 ± |
| | | | | | 1094.09 | 113.20 |
| SHH-24 | 2.5 | 0.26 | 0 | 0.5 | 1132.29 ± | 148.415 ± |
| | | | | | 264.698 | 37.314 |
| SHH-25 | 3.2 | 0.26 | 0 | 0.5 | 2949.83 ± | 349.275 ± |
| | | | | | 231.428 | 44.781 |
| SHH-26 | 3.85 | 0.26 | 0 | 0.5 | 3782.2 ± | 482.625 ± |
| | | | | | 68.024 | 7.8276 |
| SHH-27 | 3.2 | 0.5 | 0 | 0.5 | 4268.79 ± | 645 ± |
| | | | | | 2196.42 | 307.90 |
| SHH-28 | 4.47 | 0.26 | 0 | 0.25 | 24636.4 ± | 3690.53 ± |
| | | | | | 3381.63 | 626.04 |

Fabricating SHH Hydrogels Using Commercially Available Polyethylenimine, Branched (PEI) (M_{w}~25,000 g/Mol) and Mechanical Characterizations of these Hydrogels Using a Rheometer

Hydrogel fabrication description for SHH-33: first a 5 wt % solution of CNS in water, 2 wt % solution of CB[7], and a 2.5 wt % solution of copolymer PEI in water were prepared. This was followed by the preparation of a 3.2 wt % CNS, 0.26 wt % CB[7], and 0.25 wt % PEI mixture (Table 5) by adding 1.28 mL of CNS solution, 0.26 mL of DIW, and 0.26 mL of CB[7] solution. After using the vortex for 30 s and waiting 10 min, 0.2 mL of PEI solution was added to achieve a final sample amount of 2 mL. The solution was vortexed for another 30 s to achieve complete dissolution. Within 5 min, the mixture formed a hydrogel.

The rheological properties of PEI containing SHH hydrogels were measured at 25 °C using a rheometer (TA Instruments. Discovery HR 20 Rheometer, New Castle, DE) fitted with parallel plates (diameter 20 mm). The gap at the apex of the para-plate was set to 1 mm. The samples (≈15 mm dia.≈3 mm thickness) were placed between the para-plate and the platform with special care to avoid water evaporation. Dynamic frequency sweep tests (0.1-80 rad/s) at a fixed strain of 0.5% were measured to determine the storage modulus (G′) and loss modulus (G″) of hydrogels (Table 5). At least two samples of each hydrogel were tested for rheological tests.

TABLE 5
Compositions and rheological test results averages for PEI containing SHH hydrogels.

| | CNS | CB[7] | SPA | PEI | | |
|---|---|---|---|---|---|---|
| Sample | (wt./v) | (wt./v) | (wt./v) | (wt./v) | G′ (1) | G″ (2) |
| Units | % | % | % | % | (Pa) | (Pa) |
| SHH-33 | 3.2 | 0.26 | 0 | 0.25 | 2261.25 ± 781.56 | 314.79 ± 82.049 |

### Example 3

### On-Demand Dissolution Tests of SHH and Control Hydrogels Using Phosphate-Buffered Saline 1×(PBS)

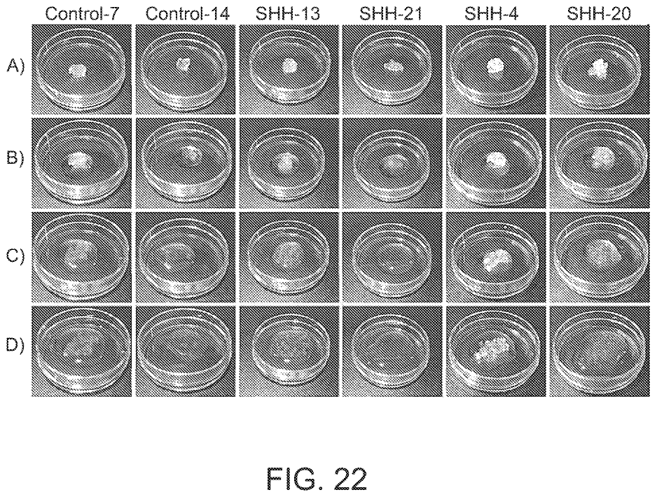

A PBS 1× solution (pH 7.4) was prepared. Approximately 0.1 g of each hydrogel was placed onto a Petri dish (FIG. 22A). The hydrogels were initially exposed to 0.2 mL of the PBS 1× solution (FIG. 22B) for 2 min and gently tapped to simulate a wound cleaning procedure. After these initial 2 min, the hydrogels were exposed to an additional 0.2 mL of the PBS 1× solution (FIG. 22C) and again gently tapped. After 4 min from the initial exposure final pictures were taken (FIG. 22D).

Hydrogels containing PMPC showed a faster and more complete dissolution when compared to PAM hydrogels. When SPA was not present, SHH-21 showed a complete dissolution after 4 min. This is likely due to the neutral character of PMPC monomeric units. The interactions between MPC units and CB7 molecules can be easily disrupted by high ionic strength solutions. SPA is a negatively charged polymer that can bridge between CNS structures and keep some level of integrity of the hydrogel at high ionic strength solutions. This is especially the case when utilizing PAM, which contains cationic charges.

### Example 4

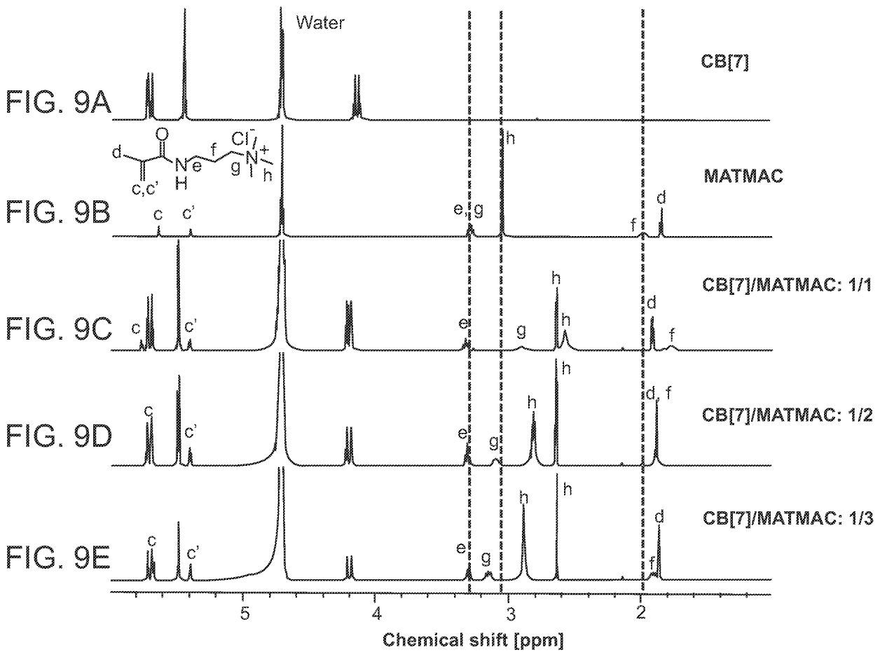

Am-r-MATMAC CP was designed to be a water-soluble copolymer that combined hydrophilic acrylamide (Am) segments with positively charged quaternary amine MATMAC segments. Each CB[7] group binds with cationic groups of Am-r-MATMAC CP through interaction and creates supramolecular crosslinks. 1H-NMR analysis of CB[7]-MATMAC interactions was performed. FIG. 9).

Additional NMR analysis on mixtures of CB[7] and the MATMAC monomer was performed, as a simplified system to determine interactions between these groups. The monomer was used as NMR peaks of the copolymer broadened, as expected in such low diffusivity systems. Peaks associated with MATMAC showed significant peak shifts, indicating a close association between MATMAC and CB[7] through partial electrostatic interactions (FIG. 4). We can see shifts in the peaks of hydrogens surrounding the cationic charge. These shifts are best described as shielding effects, most likely caused by steric effects and close proximity to other rigid chemical structures, in this case CB[7]. These shifts were observed in mixtures containing up to 3 MATMAC molecules per CB[7], indicating at least 2 and potentially more MATMAC molecules can associate with CB[7], which would lead to cross-linking. Interestingly, chemical shifts in 1:1 CB[7]:MATMAC mixtures were quite different than those observed in 1:2 and 1:3, implying different arrangements for different ratios. Previous literature shows various ways that CB[7] can interact with similar molecules ranging from the insertion of the hydrophobic groups inside the core of the ring (1:1 ratio)¹ and binding and association of amine groups with CB[7] as well as other CBs (1:2 ratio)². while our results imply that MATMAC molecules can potentially form both types of arrangements. The peaks for protons further from the quaternary amine group are not shifted as much. This makes it unlikely that the resultant structure is an inclusion complex where the whole group threads through the macrocycle; it appears the quaternary amine groups interact with the rim of the macrocycle, with at least two MATMAC molecules capable of strongly binding with each CB[7], indicated by peak shifts for associated protons. The interactions between CB[7] and MATMAC enable the supramolecular cross-linking of the CP.

Cationic Am-r-MATMAC CPs also bind with the SPA-coated clay through electrostatic interactions to form a well-dispersed, homogeneous nanocomposite that leads to enhanced mechanical properties^{33,35-37,43,44,47} (FIG. 1B). Even without SPA, electrostatic interactions can improve interactions between the CP and the CNS, improving homogeneity and stability.

This CP also includes highly hydrophilic, biocompatible repeat units—coming from the acrylamide groups—to further improve the hydration and fluid uptake of the hydrogel. Am-r-MATMAC CP was synthesized by free radical copolymerization. The synthesized CP, Am-r-MATMAC was found to contain 46 wt. % MATMAC using ¹H-NMR spectroscopy in DMSO-d₆. The ¹H-NMR spectrum of the Am-r-MATMAC CP used in this study is presented in FIG. 8 along with peak assignments. Each MATMAC unit was associated with nine protons appearing around 3 ppm and 3.1 ppm (h). The peaks around 1.9 ppm (f), 3.2 ppm (g), 3.3 ppm (e) were attributed to the CH₂ protons from MATMAC. The peak at 1.5 ppm (a) was assigned to the CH₂ protons from Am polymer backbone, whereas the peak at 1.7 ppm (c) was assigned to the CH₂ protons from MATMAC polymer.

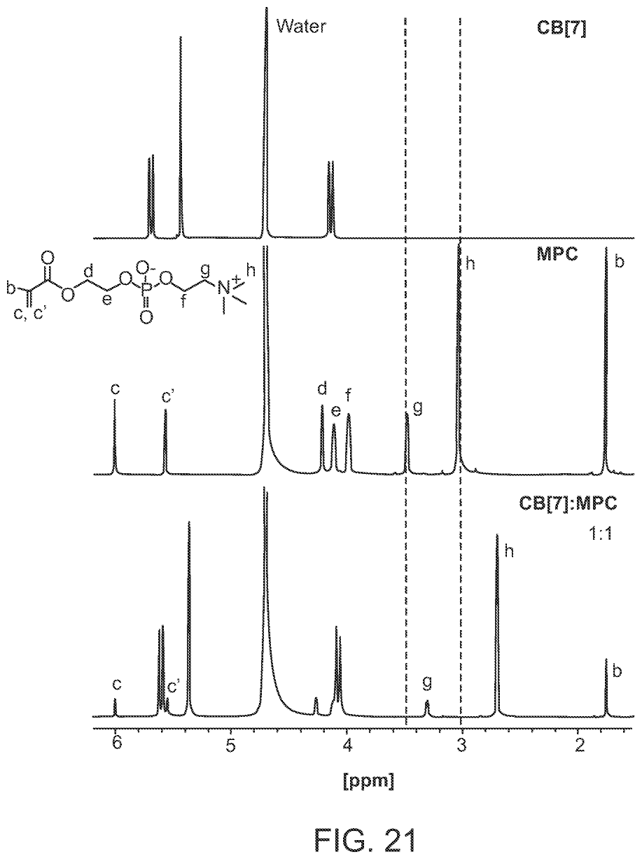

1H-NMR analysis was also performed on mixtures of CB[7] and the MPC monomer, as a simplified system to determine interactions between these groups. We observed that peaks associated with MPC showed significant peak shifts, indicating a close association between MPC and CB[7] through partial electrostatic interactions (FIG. 21). We can see shifts in the peaks of hydrogens surrounding the cationic charge. These shifts are best described as shielding effects, most likely caused by steric effects and close proximity to other rigid chemical structures, in this case CB[7] (FIG. 21).

### REFERENCES CITED

1 Forjuoh, S. N. Burns in low- and middle-income countries: a review of available literature on descriptive epidemiology, risk factors, treatment, and prevention. Burns 32, 529-537, doi:10.1016/j.burns.2006.04.002 (2006).
2 Peck, M. D., Kruger, G. E., van der Merwe, A. E., Godakumbura, W. & Ahuja, R. B. Burns and fires from non-electric domestic appliances in low and middle income countries Part I. The scope of the problem. Burns 34, 303-311, doi:10.1016/j.burns.2007.08.014 (2008).
3 Peck, M. D. Epidemiology of burns throughout the world. Part I: Distribution and risk factors. Burns 37, 1087-1100, doi:10.1016/j.burns.2011.06.005 (2011).
4 Lachiewicz, A. M., Hauck, C. G., Weber, D. J., Cairns, B. A. & van Duin, D. Bacterial Infections After Burn Injuries: Impact of Multidrug Resistance. Clin Infect Dis 65, 2130-2136, doi:10.1093/cid/cix682 (2017).
McDermott, K. W., Weiss, A. J. & Elixhauser, A. in Healthcare Cost and Utilization Project (HCUP) Statistical Briefs (2006).
6 Field, F. K. & Kerstein, M. D. Overview of wound healing in a moist environment. Am J Surg 167, 2S-6S, doi:10.1016/0002-9610(94)90002-7 (1994).
7 James, D. L. & Jowza, M. Principles of Burn Pain Management. Clin Plast Surg 44, 737-747, doi:10.1016/j.cps.2017.05.005 (2017).
8 Griggs, C., Goverman, J., Bittner, E. A. & Levi, B. Sedation and Pain Management in Burn Patients. Clin Plast Surg 44, 535-540, doi:10.1016/j.cps.2017.02.026 (2017).
9 Latarjet, J. & Choinere, M. Pain in burn patients. Burns 21, 344-348, doi:10.1016/0305-4179(95)00003-8 (1995).
10 Alencar de Castro, R. J., Leal, P. C. & Sakata, R. K. Pain management in burn patients. Braz J Anesthesiol 63, 149-153, doi:10.1016/S0034-7094(13)70206-X (2013).
11 Konieczynska, M. D. et al. On-Demand Dissolution of a Dendritic Hydrogel-based Dressing for Second-Degree Burn Wounds through Thiol-Thioester Exchange Reaction. Angew Chem Int Ed Engl 55, 9984-9987, doi:10.1002/anie.201604827 (2016).
12 Lu, W. et al. On-Demand Dissoluble Diselenide-Containing Hydrogel. Biomacromolecules 21, 3308-3317, doi:10.1021/acs.biomac.0c00762 (2020).
13 Ghobril, C., Charoen, K., Rodriguez. E. K., Nazarian, A. & Grinstaff, M. W. A dendritic thioester hydrogel based on thiol-thioester exchange as a dissolvable sealant system for wound closure. Angew Chem Int Ed Engl 52, 14070-14074, doi:10.1002/anie.201308007 (2013).
14 Konieczynska, M. D. et al. A hydrogel sealant for the treatment of severe hepatic and aortic trauma with a dissolution feature for post-emergent care. Mater Horiz 4, 222-227, doi:10.1039/C6MH00378H (2017).
Anumolu, S. S. et al. Doxycycline hydrogels with reversible disulfide crosslinks for dermal wound healing of mustard injuries. Biomaterials 32, 1204-1217, doi:10.1016/j.biomaterials.2010.08.117 (2011).
16 Xu, X., Lu, W., Zhu, J., Pan, X. & Zhu, X. An On-Demand Dissoluble Chitosan Hydrogel Containing Dynamic Diselenide Bond. Gels 7, doi:10.3390/gels7010021 (2021).
17 Huang, W. et al. On-Demand Dissolvable Self-Healing Hydrogel Based on Carboxymethyl Chitosan and Cellulose Nanocrystal for Deep Partial Thickness Burn Wound Healing. ACS Appl Mater Interfaces 10, 41076-41088, doi:10.1021/acsami.8b14526 (2018).
18 Katherine A. Cook et al. In situ gelling and dissolvable hydrogels for use as on-demand wound dressings for burns. Biomater. Sci., 9, 6842-6850, doi:10.1039/d1bm00711d (2021).
19 Xu, W., Song, Q., Xu, J. F., Serpe, M. J. & Zhang, X. Supramolecular Hydrogels Fabricated from Supramonomers: A Novel Wound Dressing Material. ACS Appl Mater Interfaces 9, 11368-11372, doi:10.1021/acsami.7b02850 (2017).
20 Konieczynska, M. D. & Grinstaff, M. W. On-Demand Dissolution of Chemically Cross-Linked Hydrogels. Acc Chem Res 50, 151-160, doi:10.1021/acs.accounts.6b00547 (2017).
21 Anderson, J. C., Blake, A. J., Graham, J. P. & Wilson, C. Investigation of the asymmetric ionic Diels-Alder reaction for the synthesis of cis-decalins. Org Biomol Chem 1, 2877-2885, doi:10.1039/b305116a (2003).
22 Baldwin, A. D. & Kiick, K. L. Reversible maleimide-thiol adducts yield glutathione-sensitive poly(ethylene glycol)-heparin hydrogels. Polym Chem 4, 133-143, doi:10.1039/C2PY20576A (2013).
23 Kharkar, P. M., Kiick, K. L. & Kloxin, A. M. Design of Thiol- and Light-sensitive Degradable Hydrogels using Michael-type Addition Reactions. Polym Chem 6, 5565-5574, doi:10.1039/C5PY00750J (2015).
24 Lu, H. et al. Recent advances of on-demand dissolution of hydrogel dressings. Burns Trauma 6, 35, doi:10.1186/s41038-018-0138-8 (2018).
Du, X., Zhou, J., Shi, J. & Xu, B. Supramolecular Hydrogelators and Hydrogels: From Soft Matter to Molecular Biomaterials. Chem Rev 115, 13165-13307, doi:10.1021/acs.chemrev.5b00299 (2015).
26 Ma, X. & Zhao, Y. Biomedical Applications of Supramolecular Systems Based on Host-Guest Interactions. Chem Rev 115, 7794-7839, doi:10.1021/cr500392w (2015).
27 Mantooth, S. M., Munoz-Robles, B. G. & Webber, M. J. Dynamic Hydrogels from Host-Guest Supramolecular Interactions. Macromol Biosci 19, e1800281, doi:10.1002/mabi.201800281 (2019).
28 Assaf, K. I. & Nau, W. M. Cucurbiturils: from synthesis to high-affinity binding and catalysis. Chem Soc Rev 44, 394-418, doi:10.1039/c4cs00273c (2015).
29 Rekharsky, M. V. et al. A synthetic host-guest system achieves avidin-biotin affinity by overcoming enthalpy-entropy compensation. Proc Natl Acad Sci USA 104, 20737-20742, doi:10.1073/pnas.0706407105 (2007).
30 Appel, E. A. et al. Ultrahigh-water-content supramolecular hydrogels exhibiting multistimuli responsiveness. J Am Chem Soc 134, 11767-11773, doi:10.1021/ja3044568 (2012).
31 Yan, X., Wang, F., Zheng, B. & Huang, F. Stimuli-responsive supramolecular polymeric materials. Chem Soc Rev 41, 6042-6065, doi:10.1039/c2cs35091b (2012).
32 Yang, L., Tan, X., Wang, Z. & Zhang, X. Supramolecular Polymers: Historical Development, Preparation, Characterization, and Functions. Chem Rev 115, 7196-7239, doi:10.1021/cr500633b (2015).
33 Wang, Q. et at. High-water-content mouldable hydrogels by mixing clay and a dendritic molecular binder. Nature 463, 339-343, doi:10.1038/nature08693 (2010).
34 Haraguchi, K. & Takehisa, T. Nanocomposite Hydrogels: A Unique Organic-Inorganic Network Structure with Extraordinary Mechanical, Optical, and Swelling/De-swelling Properties. Adv. Mater. 14, 1120-1124 (2002).
35 Li, Z., Hou, Z., Fan, H. & Li, H. Organic-inorganic hierarchical self-assembly into robust luminescent supramolecular hydrogel. Advanced Functional Materials 27, 1604379 (2017).
36 Yang, D. et at. Color-tunable luminescent hydrogels with tough mechanical strength and self-healing ability. Journal of Materials Chemistry C 6, 1153-1159 (2018).
37 Tamesue, S. et al. Linear versus dendritic molecular binders for hydrogel network formation with clay nanosheets: studies with ABA triblock copolyethers carrying guanidinium ion pendants. J Am Chem Soc 135, 15650-15655, doi:10.1021/ja408547g (2013).
38 Garcia-Astrain, C. et a. Green chemistry for the synthesis of methacrylate-based hydrogels crosslinked through Diels-Alder reaction. Eur. Polym. J. 49, 3998-4007, doi:10.1016/j.eurpolymj.2013.09.004 (2013).
39 Yang, C. et at. A green fabrication approach of gelatin/CM-chitosan hybrid hydrogel for wound healing. Carbohydr. Polym. 82, 1297-1305, doi:10.1016/j.carbpol.2010.07.013 (2010).
40 Wang, Z. et at. Green Gas-Mediated Cross-Linking Generates Biomolecular Hydrogels with Enhanced Strength and Excellent Hemostasis for Wound Healing. ACS Appl Mater Interfaces 12, 13622-13633, doi:10.1021/acsami.9b21325 (2020).
41 Mubarok, W., Qu, Y. & Sakai, S. Influence of Hydrogen Peroxide-Mediated Cross-Linking and Degradation on Cell-Adhesive Gelatin Hydrogels. ACS Appl Bio Mater 4, 4184-4190, doi:10.1021/acsabm.0c01675 (2021).
42 Muhamed, J., Revi, D., Rajan, A., Geetha, S. & Anilkumar, T. V. Biocompatibility and Immunophenotypic Characterization of a Porcine Cholecyst-derived Scaffold Implanted in Rats. Toxicol Pathol 43, 536-545, doi:10.1177/0192623314550722 (2015).
43 Li, Z., Zhang, Y. M., Wang, H., Li, H. & Liu, Y. Mechanical behaviors of highly swollen supramolecular hydrogels mediated by pseudorotaxanes. Macromolecules 50, 1141-1146, doi:10.1021/acs.macromol.6b02459 (2017).
44 Lee, J. H., Kim, C. & Jung, J. H. Control of the rheological properties of clay nanosheet hydrogels with a guanidinium-attached calix[4]arene binder. Chem Commun (Camb) 51, 15184-15187. doi:10.1039/c5cc06024a (2015).
45 Das, D., Assaf, K. 1. & Nau, W. M. Applications of Cucurbiturils in Medicinal Chemistry and Chemical Biology. Front Chem 7, 619, doi:10.3389/fchem.2019.00619 (2019).
46 Barrow, S. J., Kasera, S., Rowland. M. J., del Barrio, J. & Scherman, O. A. Cucurbituril-Based Molecular Recognition. Chem Rev 115, 12320-12406, doi:10.1021/acs.chemrev.5b00341 (2015).
47 Bullock, A. J., Pickavance, P., Haddow, D. B., Rimmer, S. & MacNeil, S. Development of a calcium-chelating hydrogel for treatment of superficial burns and scalds. Regen Med 5, 55-64, doi:10.2217/rme.09.67 (2010).
48 Pawlaczyk, M., Lelonkiewicz, M. & Wieczorowski, M. Age-dependent biomechanical properties of the skin. Advances in Dermatology and Allergology/Postȩpy Dermatologii i Alergologii 30, 302-306 (2013).
49 Crichton, M. L., Chen, X., Huang, H. & Kendall, M. A. Elastic modulus and viscoelastic properties of full thickness skin characterised at micro scales. Biomaterials 34, 2087-2097 (2013).
50 Basti, A. T. K. et al. Employing Cellulose Nanofiber-Based Hydrogels for Burn Dressing. Polymers 14, 1207 (2022).
51 Lee, J. H., Kim, C. & Jung, J. H. Control of the rheological properties of clay nanosheet hydrogels with a guanidinium-attached calix [4] arene binder. Chemical Communications 51, 15184-15187 (2015).
52 Lagona, J., Mukhopadhyay, P., Chakrabarti, S. & Isaacs, L. The cucurbit[n]uril family. Angew Chem Int Ed Engl 44, 4844-4870, doi:10.1002/anie.200460675 (2005).
53 He, J. et al. Injectable Self-Healing Adhesive pH-Responsive Hydrogels Accelerate Gastric Hemostasis and Wound Healing. Nanomicro Lett 13, 80, doi:10.1007/s40820-020-00585-0 (2021).
54 Qu, J. et al. Antibacterial adhesive injectable hydrogels with rapid self-healing, extensibility and compressibility as wound dressing for joints skin wound healing. Biomaterials 183, 185-199, doi:10.1016/j.biomaterials.2018.08.044 (2018).
55 Chen, M. et al. Dynamic covalent constructed self-healing hydrogel for sequential delivery of antibacterial agent and growth factor in wound healing. Chemical Engineering Journal 373, 413-424 (2019).
56 Zhao, L. et al. pH and glucose dual-responsive injectable hydrogels with insulin and fibroblasts as bioactive dressings for diabetic wound healing. ACS applied materials & interfaces 9, 37563-37574 (2017).
57 Li, Z. et al. Hydrogel cross-linked with dynamic covalent bonding and micellization for promoting burn wound healing. ACS applied materials & interfaces 10, 25194-25202 (2018).
58 Taylor, D. L. & In Het Panhuis, M. Self-Healing Hydrogels. Adv Mater 28, 9060-9093, doi:10.1002/adma.201601613 (2016).
59 Zhang, B., He, J., Shi, M., Liang, Y. & Guo, B. Injectable self-healing supramolecular hydrogels with conductivity and photo-thermal antibacterial activity to enhance complete skin regeneration. Chemical Engineering Journal 400, 125994 (2020).
60 Ribeiro, M. P. et al. Development of a new chitosan hydrogel for wound dressing. Wound Repair Regen 17, 817-824, doi:10.1111/j.1524-475X.2009.00538.x (2009).
61 Ono, S. et al. Increased wound pH as an indicator of local wound infection in second degree burns. Burns 41, 820-824, doi:10.1016/j.burns.2014.10.023 (2015).
62 Kelso, A. Cytokines: principles and prospects. Immunol Cell Biol 76, 300-317, doi:10.1046/j.1440-1711.1998.00757.x (1998).
63 Anderson, J. M.. Rodriguez. A. & Chang, D. T. Foreign body reaction to biomaterials. Semin Immunol 20, 86-100, doi:10.1016/j.smim.2007.11.004 (2008).
64 Williams. D. F. On the mechanisms of biocompatibility. Biomaterials 29, 2941-2953, doi:10.1016/j.biomaterials.2008.04.023 (2008).
65 Ratner, B. D. The biocompatibility manifesto: biocompatibility for the twenty-first century. J Cardiovasc Transl Res 4, 523-527, doi:10.1007/s12265-011-9287-x (2011).
66 Boardman, D. A. et al. Expression of a Chimeric Antigen Receptor Specific for Donor HLA Class I Enhances the Potency of Human Regulatory T Cells in Preventing Human Skin Transplant Rejection. Am J Transplant 17, 931-943, doi:10.1111/ajt.14185 (2017).
67 Ratnasothy, K. et al. IL-2 therapy preferentially expands adoptively transferred donor-specific Tregs improving skin allograft survival. Am J Transplant 19, 2092-2100, doi:10.1111/ajt.15306 (2019).
68 Zhai, Q. et al. An immune-competent rat split thickness skin graft model: useful tools to develop new therapies to improve skin graft survival. Am J Transl Res 10, 1600-1610 (2018).
69 Ordikhani, F., Pothula, V., Sanchez-Tarjuelo, R., Jordan, S. & Ochando, J. Macrophages in Organ Transplantation. Front Immunol 11, 582939, doi:10.3389/fimmu.2020.582939 (2020).
70 Di Liddo, R. et al. Histone deacetylase inhibitors restore IL-10 expression in lipopolysaccharide-induced cell inflammation and reduce IL-1beta and IL-6 production in breast silicone implant in C57BL/6J wild-type murine model. Autoimmunity, 1-11, doi:10.3109/08916934.2015.1134510 (2016).
71 Price, T. H., Chatta, G. S. & Dale, D. C. Effect of recombinant granulocyte colony-stimulating factor on neutrophil kinetics in normal young and elderly humans. Blood 88, 335-340 (1996).
72 Shochat, E., Rom-Kedar, V. & Segel, L. A. G-CSF control of neutrophils dynamics in the blood. Bull Math Biol 69, 2299-2338, doi:10.1007/s11538-007-9221-1 (2007).
73 Schumacher, C., Clark-Lewis, I., Baggiolini, M. & Moser, B. High- and low-affinity binding of GRO alpha and neutrophil-activating peptide 2 to interleukin 8 receptors on human neutrophils. Proc Natl Acad Sci USA 89, 10542-10546, doi:10.1073/pnas.89.21.10542 (1992).
74 Luttikhuizen, D. T. et al. The correlation between difference in foreign body reaction between implant locations and cytokine and MMP expression. Biomaterials 27, 5763-5770, doi:10.1016/j.biomaterials.2006.07.004 (2006).

What is claimed is:

1. A hydrogel composition, comprising:
a water-soluble polymer that comprises functional groups capable of interacting with a macrocyclic ligand molecule;
a ligand molecule; and
a biocompatible nanomaterial.

2. The hydrogel composition of claim 1, further comprising a water-soluble anionic polymer.

3. The hydrogel composition of claim 1, wherein the water-soluble polymer comprises cationic nitrogen-containing groups.

4-10. (canceled)

11. The hydrogel composition of claim 3, wherein the water-soluble polymer is polyacrylamide-random-[3-(methacryloylamino)propyl]trimethylammonium chloride, poly(2-methacryloyloxyethyl phosphorylcholine), polyethylenimine (branched), polyethylenimine (linear), poly(2-dimethylamino)ethyl methacrylate) methyl chloride quaternary salt, polydiallyldimethylammonium chloride, poly[bis(2-chloroethyl) ether-alt-1,3-bis[3-(dimethylamino)propyl]urea]quaternized, poly(2-(dimethylamino)ethyl methacrylate), poly(2-(diethylamino)ethyl methacrylate), poly(2-(diisopropylamino)ethyl methacrylate), poly(allylamine hydrochloride), poly(acrylamide-co-diallyldimethylammonium chloride), poly(L-lysine hydrobromide), or poly[(2-ethylmethylaminoethyl methacrylate salt)-co-(1-vinylpyrrolidone).

12. The hydrogel composition of claim 1, wherein the biocompatible nanomaterial is selected from exfoliated clay nanosheets, nanocellulose-based materials (e.g., nanowhiskers, nanofibers, nanocrystals, bacterial nanocellulose), chitosan- and chitin-based nanomaterials, starch-based nanomaterials, ceramic nanoparticles (e.g., silica, mesoporous silica, hydroxyapatite, aluminum oxide, zinc oxide, iron oxide, titanium dioxide), metallic nanoparticles (e.g., silver, gold), graphene oxide, and carbon nanotubes.

13. The hydrogel composition of claim 1, wherein the ligand molecule is selected from a crown ether, cucurbit[n]uril, calix[n]arene, pillar[n]arene and cyclodextrin.

14. (canceled)

15. (canceled)

16. The hydrogel composition of claim 13, wherein the ligand molecule is cucurbit[7]uril.

17. (canceled)

18. The hydrogel composition of claim 1, comprising:
polyacrylamide-random-[3-(methacryloylamino)propyl]trimethylammonium chloride;
cucurbit[7]uril; and
exfoliated clay nanosheets.

19.-33. (canceled)

34. The hydrogel composition of claim 1, wherein the composition further comprises an antimicrobial agent.

35-39. (canceled)

40. A wound dressing, comprising the hydrogel composition of claim 1.

41. (canceled)

42. A method of treating a wound in subject in need thereof, comprising the steps of:
contacting a first solution comprising a water-soluble polymer that comprises functional groups that are capable of interacting with a macrocyclic ligand molecule with a second solution comprising a ligand molecule to generate a hydrogel; and
applying the hydrogel to the wound.

43. The method of claim 42, wherein the first solution or the second solution further comprises a biocompatible nanomaterial.

44. The method of claim 43, wherein the first solution or the second solution further comprises a water-soluble anionic polymer.

45.-52. (canceled)

53. The method of claim 42, wherein the water-soluble polymer is polyacrylamide-random-[3-(methacryloylamino)propyl]trimethylammonium chloride, poly(2-methacryloyloxyethyl phosphorylcholine) polyethylenimine (branched), polyethylenimine (linear), poly(2-dimethylamino)ethyl methacrylate) methyl chloride quaternary salt, polydiallyldimethylammonium chloride, poly[bis(2-chloroethyl) ether-alt-1,3-bis[3-(dimethylamino)propyl]urea]quaternized, poly(2-(dimethylamino)ethyl methacrylate), poly(2-(diethylamino)ethyl methacrylate), poly(2-(diisopropylamino)ethyl methacrylate), poly(allylamine hydrochloride), poly(acrylamide-co-diallyldimethylammonium chloride), poly(L-lysine hydrobromide), or poly[(2-ethylmethylaminoethyl methacrylate salt)-co-(1-vinylpyrrolidone).

54. (canceled)

55. The method of claim 42, wherein the ligand molecule is selected from a crown ether, cucurbit[n]uril, calix[n]arene, pillar[n]arene and cyclodextrin.

56. (canceled)

57. (canceled)

58. The method of claim 55, wherein the ligand molecule is cucurbit[7]uril.

59. (canceled)

60. The method of claim 42 wherein the hydrogel comprises:
polyacrylamide-random-[3-(methacryloylamino)propyl]trimethylammonium chloride;
cucurbit[7]uril; and
exfoliated clay nanosheets.

61.-86. (canceled)

87. The method of claim 42, further comprising the step of removing the hydrogel from the wound by dissolution by applying a third solution.

88.-90. (canceled)

91. The method of claim 87, wherein the third solution comprises a dissolution agent having a structure of Formula (I) or (II): 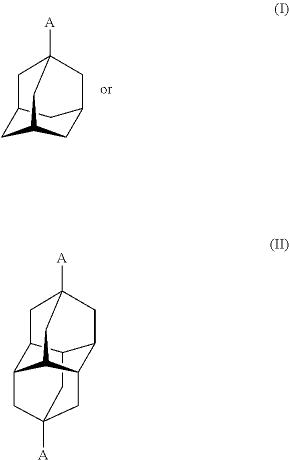
(I) or A (II) with a counterion X⁻;
wherein:
each A is independently selected from —N(R¹)₃⁺, —(CH₂)ₘN(R¹)₃⁺ and —NH(CH₂)ₙN(R¹)₃⁺; wherein,
each R¹ is independently selected from H and C₁-C₄ alkyl;
m is 1 to 6;
n is 2 to 6; and
X⁻ is a pharmaceutically acceptable counterion.

92-102. (canceled)

103. The method of claim 42, wherein
the wound is a burn.

104.-105. (canceled)
